(19) United States
(12) Patent Application Publication
(10) Pub. No.: US 2013/0074215 A1
(43) Pub. Date: Mar. 21, 2013

(54) MODULATION OF GALACTOMANNAN CONTENT IN COFFEE

(71) Applicants: James Gerard McCarthy, Noizay (FR); Maud Nicole Claire Leppelley, Saint-Cyr-sur-Loire (FR)

(72) Inventors: James Gerard McCarthy, Noizay (FR); Maud Nicole Claire Leppelley, Saint-Cyr-sur-Loire (FR)

(73) Assignee: NESTEC S.A., Vevey (CH)

(21) Appl. No.: 13/583,433

(22) PCT Filed: Mar. 7, 2011
(86) PCT No.: PCT/EP2011/053352

§ 371 Date: Sep. 7, 2012

(30) Foreign Application Priority Data
Mar. 9, 2010 (EP) 10155941.7

(51) Int. Cl. C12N 15/82; C07H 21/04

(52) U.S. Cl. CPC C12N 15/8242
USPC 800/278; 536/23.2; 435/320.1; 800/298

(57) ABSTRACT

Disclosed herein are nucleic acid molecules isolated from coffee (Coffea spp.) comprising sequences that encode UDP-glucose pyrophosphorylase (UGPP), GDP-mannose pyrophosphorylase (GMPP), phosphomannomutase (PMM), and UDP-glucose 4-epimerase (UGE). Also disclosed are methods for using these polynucleotides for gene regulation and manipulation of the content and/or structure of coffee grains, to influence extraction characteristics and other features.

### FIELD OF THE INVENTION

The present invention relates to the field of agricultural biotechnology. More particularly, the invention relates to nucleic acids and enzymes from coffee plants that are involved in the synthesis of galactomannan precursors, and their use in modulating galactomannan content of coffee beans.

### BACKGROUND OF THE INVENTION

Plant cell walls are complex and dynamic composites comprising, especially, polysaccharides, proteins, and lignin. Polysaccharides are major constituents of the green coffee grain, representing up to 50% of the dry weight of the mature grain (Redgwell et al., 2003, Planta 217, 316-326). There are three major forms of polysaccharides in the coffee grain: cellulose, arabinogalactan type II and galactomannans (Fischer et al., 2001, Carbohydrate Research 330, 93-101), with the galactomannans being the most abundant (50% of the total, or approximately 25% of the dry mass of the grain). The galactomannan structure is relatively simple, consisting of a linear backbone of β-1,4-linked mannose molecules with single-unit α-1, 6-linked galactosyl side chains at various intervals along the mannan backbone. In some plants, though it is not certain for coffee, there is also glucose interspersed with mannans, generating galactoglucomannans.

Clifford (1986, Tea Coffee Trade J. 158, 30-32) has reported that arabica coffee (Coffea arabica) may contain more arabinogalactans (9-13%) than robusta (C. canephora) (6-8%) and as well as more galactomannan (25-30% in arabica vs. 19-22% in robusta). He also suggested that the galactomannans in robusta are more highly branched and thus less crystalline. This proposition, though not yet substantiated, has been used to explain why, at the same degree of roasting, robustas generally produce more soluble solids than arabicas (Clifford, 1985, In: M. N. Clifford and K. C. Wilson, Editors, Coffee Botany, Biochemistry, and Production of Beans and Beverage, Croom Helm, London, pp. 305-374.; Clifford, 1986, supra; Trugo, 1985, In: R. J. Clarke and R. Macrae, Editors, Coffee, Chemistry 1, Elsevier, Amsterdam, pp. 83-114). It is noted that in a study of the polysaccharides isolated from the grain of one arabica variety and one robusta variety, Fischer et al. (2001) did not find any significant differences in the amount of polysaccharides in those samples, although the polysaccharides of the arabica variety had slightly more mannose content than did the robusta variety. In addition, no detectable differences were seen in the galactomannans of the arabica and robusta varieties examined. Redgwell et al., 2002, Carbohydrate Research 337, 421-431) and Oosterveld et al., 2003, Carbohydrate Polymers 52, 285-296) have reported that more than 40% of polysaccharides originally present in the green grain are degraded after longer periods of roasting. However, Redgwell et al. (2002, supra) showed that the arabinogalactans are more susceptible to degradation during roasting than the mannans, and that the cellulose polymers were unaffected. The limited degradation of the galactomannans during roasting has led to the idea that galactomannans are among the most difficult parts of the roasted grain to extract. As the galactomannans make up a major portion of the coffee grain, a significant amount of research effort has been employed to understand how galactomannans are synthesized and degraded in the coffee grain endosperm. The main objective of that research has been to find and/or develop coffee with altered galactomannan levels and/or structure, and thus which may, for example, have improved extractability at lower temperatures.

Galactomannan synthesis is carried out by two enzymes, mannan synthase (ManS) and galactomannan galactosyltransferases (GMGT), which are though to be co-localized in the membrane of Golgi vesicules and are believed to work together as a complex (Dhugga et al., 2004, Science 303, 363-366; Edwards et al., 2004, Plant Physiol 134, 1153-1162). The ManS and GMGT gene sequences involved in coffee grain galactomannan synthesis, as well as sequences for galactomannan synthesis in other parts of the plant, have been isolated and characterized (Pre et al., 2008, Ann. Bot. (Lond) 102, 207-220; WO 2007/047675 A2). During galactomannan synthesis, the ManS enzyme, which is related to the cellulose synthetases, polymerizes the mannan backbone using the GDP-Mannose (GDP-Man) precursors, while the GMGT enzyme is responsible for transferring galactose residues from the UDP-Galactose precursor to the growing mannan backbone (Edwards et al., 2004, supra). It was suggested that modulating the expression or activity of the enzymes encoded by those genes could be used to increase or decrease the amount of galactomannan in the plant, most particularly in the bean (WO 2007/047675 A2).

Mannanases are involved in galactomannan degradation, which can also affect the amount of galactomannan present in a plant or plant tissue. Coffee mannanases have been isolated and characterized (WO 00/28046; U.S. Pat. No. 7,148,399 B2). Two cDNA encoding distinct endo-beta mannanases (manA and manB) have been isolated from germinating coffee grain (Marraccini et al., 2001, Planta 213: 296-308).

Despite the significance of galactomannans in coffee grain and the implicit importance of enzymes that participate in galactomannan synthesis and degradation, little progress has been made in modulating either the amount or structure of galactomannans in the grain, through the use of those genes or enzymes. Thus a need exists to identify and develop new ways to manipulate galactomannan content and/or structure in coffee.

### SUMMARY OF THE INVENTION

One aspect of the present invention features a nucleic acid molecule isolated from Coffea spp. comprising a coding sequence that encodes a galactomannan precursor synthesis enzyme selected from UDP-glucose pyrophosphorylase (UGPP), GDP-mannose pyrophosphorylase (GMPP), phosphomannomutase (PMM), and UDP-glucose 4-epimerase (UGE). In one embodiment, the galactomannan precursor synthesis enzyme comprises an amino acid sequence greater than about 80% identical across its entirety to that of any one of SEQ ID NOs: 6-10, as determined by BLAST comparison. In particular, the galactomannan precursor synthesis enzyme comprises any one of SEQ ID NOs: 6-10. The nucleic acid molecule may comprise any one of SEQ ID NOs: 1-5. In certain embodiments, the coding sequence is (1) an open reading frame of a gene, or (2) an mRNA molecule produced by transcription of the gene, or (3) a cDNA molecule produced by reverse transcription of the mRNA molecule.

Another aspect of the invention features a vector comprising the aforementioned coding sequence that encodes a galactomannan precursor synthesis enzyme selected from UDP-glucose pyrophosphorylase (UGPP), GDP-mannose pyrophosphorylase (GMPP), phosphomannomutase (PMM), and UDP-glucose 4-epimerase (UGE). In one embodiment, the vector is an expression vector selected from the group of vectors consisting of plasmid, phagemid, cosmid, baculovirus, bacmid, bacterial, yeast and viral vectors. The coding sequence of the nucleic acid molecule can be operably linked to a constitutive promoter, or it can be operably linked to an inducible promoter, or it can be operably linked to a tissue specific promoter. Some promoters are both inducible and tissue specific, while others are constitutive and tissue specific. Optionally, the tissue specific promoter is a seed specific promoter. Seed specific promoters from coffee are particularly suitable.

Another aspect of the invention features a host cell transformed with one or more of the aforementioned vectors. The host cell may be selected from selected from plant cells, bacterial cells, fungal cells, insect cells and mammalian cells. In certain embodiments, the host cell is a plant cell selected from the group of plants consisting of coffee, tobacco, Arabidopsis, maize, wheat, rice, soybean barley, rye, oats, sorghum, alfalfa, clover, canola, safflower, sunflower, peanut, cacao, tomatillo, potato, pepper, eggplant, sugar beet, carrot, cucumber, lettuce, pea, aster, begonia, chrysanthemum, delphinium, petunia, zinnia, and turfgrasses. In a particular embodiment, the host cell is from coffee. A fertile plant produced from any of the foregoing the plant cells is also provided.

Another aspect of the invention features method of modulating galactomannan content in a plant, comprising modulating production or activity of one or more galactomannan precursor synthesis enzymes within the plant, to result in altered galactomannan content of the plant. In particular embodiments, the plant is a coffee plant. Such methods can be used to modulate the extractability of coffee seeds by altering the amount and/or structure of galactomannan within the coffee seeds. In certain embodiments, the galactomannan precursor synthesis enzyme is UDP-glucose pyrophosphorylase (UGPP), GDP-mannose pyrophosphorylase (GMPP), phosphomannomutase (PMM), or UDP-glucose 4-epimerase (UGE).

One embodiment of the method comprises increasing production or activity of one or more of the UGPP, GMPP, PMM, or UGE, for example by increasing expression of a gene encoding one or more of the UGPP, GMPP, PMM, or UGE within the plant. This can be accomplished by introducing one or more transgenes encoding one or more of the UGPP, GMPP, PMM, or UGE into the plant.

Another embodiment of the method comprises decreasing production or activity of one or more of the UGPP, GMPP, PMM, or UGE, for example, by decreasing expression of a gene encoding one or more of the UGPP, GMPP, PMM, or UGE within the plant. This may be accomplished by introducing into the plant one or more polynucleotides encoding an inhibitor of translation of one or more of the UGPP, GMPP, PMM, or UGE, such as an antisense oligonucleotide, siRNA, miRNA or shRNA.

Other features and advantages of the invention will be understood by reference to the drawings, detailed description and examples that follow.

### BRIEF DESCRIPTION OF THE DRAWINGS

FIG. 1. Protein sequence alignment of CcUGPP (pcccs46w918) (SEQ ID NO:6), StUGPP (CAA79357) (SEQ ID NO:11), OsUGPP (ABD57308) (SEQ ID NO:12), CmUGPP (ABD98820) (SEQ ID NO:13) and AtUGPP (AAK32773) (SEQ ID NO:14). An alignment of Solanum tuberosum UGPP (StUGPP), Oryza sativa UGPP (OsUGPP), Cucumis melo UGPP (CmUGPP) and Arabidopsis thaliana UGPP (AtUGPP) protein sequences available in the NCBI database with the protein sequence encoded by Coffea canephora UGPP gene (CcUGPP) was done using CLUSTAL W. Amino acids marked in gray match the residues found in Coffea canephora UGPP sequence.

FIG. 2. Protein sequence alignment of CcGMPP (pccc122i19) (SEQ ID NO:7), StGMPP (AAD01737) (SEQ ID NO:15), S1GMPP (AAT37498) (SEQ ID NO:16), MsGMPP (AAT58365) (SEQ ID NO:17) and VvGMPP (CA069137) (SEQ ID NO:18). An alignment of Solanum tuberosum GMPP (StGMPP), Solanum lycopersicum GMPP (S1GMPP), Medicago sativa GMPP (MsGMPP) and Vitis vinifera GMPP (VvGMPP) protein sequences available in the NCBI database with the protein sequence encoded by Coffea canephora GMPP gene (CcGMPP) was done using CLUSTAL W. Amino acids marked in gray match the residues found in Coffea canephora GMPP sequence.

FIG. 3. Protein sequence alignment of CcPMM (pcccs46w3a14) (SEQ ID NO:8), GmPMM (ABD97873) (SEQ ID NO:19), VvPMM (CA039354) (SEQ ID NO:20), PtPMM (ABK96056) (SEQ ID NO:21) and AtPMM (ABD97870) (SEQ ID NO:22). An alignment of Glycine max PMM (GmPMM), Vitis vinifera PMM (VvPMM), Populus trichocarpa PMM (PtPMM) and Arabidopsis thaliana PMM (AtPMM) protein sequences available in the NCBI database with the protein sequence encoded by Coffea canephora PMM gene (CcPMM) was done using CLUSTAL W. Amino acids marked in gray match the residues found in Coffea canephora PMM sequence.

FIG. 4. Protein sequence alignment of CcUGE1 (SGN-U347952) (SEQ ID NO:9), CcUGE5 (pccc117j24) (SEQ ID NO:10), AtUGE1 (NP_{—}172738) (SEQ ID NO:23), AtUGE3 (NP_{—}564811) (SEQ ID NO:24), StUGE51 (AAP97493) (SEQ ID NO:25), AtUGE5 (NP_{—}192834) (SEQ ID NO:26), AtUGE2 (NP_{—}194123) (SEQ ID NO:27), AtUGE4 (NP_{—}176625) (SEQ ID NO:28) PtUGE (ABK95303) (SEQ ID NO:29), StUGE45 (AAP42567) (SEQ ID NO:30) and VvUGE (CAN63477) (SEQ ID NO:31). An alignment of Arabidopsis thaliana UGE1 (AtUGE1), UGE3 (AtUGE3), Solanum tuberosum UGE51 (StUGE51), Populus trichocarpa UGE (PtUGE), Solanum tuberosum UGE45 (StUGE45) and Arabidopis thaliana UGE2 (AtUGE2), UGE4 (AtUGE4) and UGE5 (AtUGE5) protein sequences available in the NCBI database with the protein sequences encoded by Coffea canephora UGE1 (CcUGE1) and UGE5 (CcUGE5) genes was done using CLUSTAL W. Amino acids marked in gray match the residues found in Coffea canephora UGE1 sequence.

FIG. 5. Phylogenetic tree obtained by MegAlign software deriving from the proteic alignment performed using ClustalW represented in FIG. 4.

FIG. 6. Expression of the recombinant His-Tagged CcUGE5 and CcUGPP. Extracts from various stages of the expression of the recombinant HIS-CcUGE5 and HIS-CcUGPP fusion proteins (pGT2 and pGT3, respectively) were analyzed on a 8-16% Acrylamide Express PAGE Gel (GenScript Corp.) using coomassie blue staining. The ladder was deposited in the left of the gel (Prestained SDS-PAGE Standards Low Range (BIO-RAD)). For each protein, four frations were deposited and are (from the left to the right):

Non induced: Total lysate of B121 recombinant cells containing pGT2 (HIS-CcUGE5) or pGT3 (CcUGPP) not induced; Induced: Total lysate of B121 recombinant cells containing pGT2 (HIS-CcUGE5) or pGT3 (CcUGPP) induced with 0.2 mM IPTG; Soluble: soluble fraction of induced lysate after lysis treatment using the BugBuster; Insoluble: insoluble fraction of induced lysate after lysis treatment using the BugBuster.

FIG. 7. Quantitative expression analysis of UGE1 and UGE5 at different grain development stages for robusta FRT32, FRT05 and FRT64 and arabica T2308. The expression of each gene was measured in the various grain samples using quantitative RT-PCR. RQ is the expression level of the gene relative to the constitutively expressed gene RPL39. SG, small green stage grain; LG, large green stage grain; YG, yellow stage grain; RG, red stage grain.

The codes of the cDNA used is this experiment are: cDNA3-RNA FRT32-1, cDNA1-RNA FRT05-3, cDNA1-RNA FRT64-3 and cDNA3-RNA T2308-2.

FIG. 8. Quantitative expression analysis of UGPP, GMPP and PMM at different grain development stages for robusta FRT32, FRT05 and FRT64 and arabica T2308. The expression of each gene was measured in the various grain samples using quantitative RT-PCR. RQ is the expression level of the gene relative to the constitutively expressed gene RPL39. SG, small green stage grain; LG, large green stage grain; YG, yellow stage grain; RG, red stage grain. The code of the cDNA used is this experiment are: cDNA3-RNA FRT32-1, cDNA1-RNA FRT05-3, cDNA1-RNA FRT64-3 and cDNA3-RNA T2308-2.

FIG. 9. Quantitative expression analysis of UGE1, UGE5, UGPP, GMPP and PMM in C. canephora (robusta, FRT32) and C. arabica (arabica, T2308). The expression of each gene was determined by quantitative RT-PCR using TaqMan specific probes as described in the methods. The RQ value for each tissue sample was determined by normalizing the transcript level of the test gene versus the transcript level of the ubiquitously expressed rp139 gene in each sample analyzed. The data represent mean values obtained from three amplification reactions for each sample and the error bars indicate the SD. G, Grain; P, Pericarp; SG, small green stage grain; LG, large green stage grain; YG, yellow stage grain; RG, red stage grain. The code of the cDNA used is this experiment are: cDNA3-RNA FRT32-1; cDNA1-RNA FRT05-3; cDNA1-RNA FRT64-3 and cDNA3-RNA T2308-2.

### DETAILED DESCRIPTION OF ILLUSTRATIVE EMBODIMENTS

### Definitions

Various terms relating to the biological molecules and other aspects of the present invention are used through the specification and claims. The terms are presumed to have their customary meaning in the field of molecular biology and biochemistry unless they are specifically defined otherwise herein.

“Isolated” means altered “by the hand of man” from the natural state. If a composition or substance occurs in nature, it has been “isolated” if it has been changed or removed from its original environment, or both. For example, a polynucleotide or a polypeptie naturally present in a living plant or animal is not “isolated,” but the same polynucleotide or polypeptide separated from the coexisting materials of its natural state is “isolated”, as the term is employed herein.

“Polynucleotide”, also referred to as “nucleic acid molecule”, generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from natural posttranslational processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., pp 1-12 in Posttranslational Covalent Modification of Proteins, B. C. Johnson, Ed., Academic Press, New York, 1983; Seifter et al., 1990, Meth Enzymol 182, 626-646 and Rattan et al., 1992, Ann NY Acad Sci 663, 48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions or deletions in any combination. A substituted or inserted amino acid residue may or may not be one encoded by the genetic code. A variant of a polynucleotide or polypeptide may be naturally occurring, such as an allelic variant, or it may be a variant that is not known to occur naturally. Non-naturally occurring variants of polynucleotides and polypeptides may be made by mutagenesis techniques or by direct synthesis.

“Antibodies” as used herein includes polyclonal and monoclonal antibodies, chimeric, single chain, and humanized antibodies, as well as antibody fragments (e.g., Fab, Fab′, F(ab′)₂ and Fᵥ), including the products of a Fab or other immunoglobulin expression library. With respect to antibodies, the term, “immunologically specific” or “specific” refers to antibodies that bind to one or more epitopes of a protein of interest, but which do not substantially recognize and bind other molecules in a sample containing a mixed population of antigenic biological molecules. Screening assays to determine binding specificity of an antibody are well known and routinely practiced in the art. For a comprehensive discussion of such assays, see Harlow et al. (Eds.), ANTIBODIES A LABORATORY MANUAL; Cold Spring Harbor Laboratory; Cold Spring Harbor, N.Y. (1988), Chapter 6.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, such as an amino acid or polypeptide, when the sequence is expressed. The coding sequence may comprise untranslated sequences (e.g., introns or 5′ or 3′ untranslated regions) within translated regions, or may lack such intervening untranslated sequences (e.g., as in cDNA). In certain public databases, e.g., GenBank, the term “CDS” is sometimes utilized. A CDS in that context is a sequence of nucleotides that corresponds with the sequence of amino acids in the encoded protein. A typical CDS starts with ATG and ends with a stop codon. The term CDS can also be used to refer to the complete coding sequence of a cDNA. The term “coding sequence” is sometimes used interchangeably with the term “open reading frame”.

“Intron” refers to polynucleotide sequences in a nucleic acid that do not code information related to protein synthesis. Such sequences are transcribed into mRNA, but are removed before translation of the mRNA into a protein.

The term “operably linked” or “operably inserted” means that the regulatory sequences necessary for expression of the coding sequence are placed in a nucleic acid molecule in the appropriate positions relative to the coding sequence so as to enable expression of the coding sequence. By way of example, a promoter is operably linked with a coding sequence when the promoter is capable of controlling the transcription or expression of that coding sequence. Coding sequences can be operably linked to promoters or regulatory sequences in a sense or antisense orientation. The term “operably linked” is sometimes applied to the arrangement of other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA” or “transgene”. Such a nucleic acid construct may contain a coding sequence for a gene product of interest, along with a selectable marker gene and/or a reporter gene.

A “marker gene” or “selectable marker gene” is a gene whose encoded gene product confers a feature that enables a cell containing the gene to be selected from among cells not containing the gene. Vectors used for genetic engineering typically contain one or more selectable marker genes. Types of selectable marker genes include (1) antibiotic resistance genes, (2) herbicide tolerance or resistance genes, and (3) metabolic or auxotrophic marker genes that enable transformed cells to synthesize an essential component, usually an amino acid, which the cells cannot otherwise produce.

A “reporter gene” is also a type of marker gene. It typically encodes a gene product that is assayable or detectable by standard laboratory means (e.g., enzymatic activity, fluorescence).

The term “express,” “expressed,” or “expression” of a gene refers to the biosynthesis of a gene product. The process involves transcription of the gene into mRNA and then translation of the mRNA into one or more polypeptides, and encompasses all naturally occurring post-translational modifications.

“Endogenous” refers to any constituent, for example, a gene or nucleic acid, or polypeptide, that can be found naturally within the specified organism.

A “heterologous” region of a nucleic acid construct is an identifiable segment (or segments) of the nucleic acid molecule within a larger molecule that is not found in association with the larger molecule in nature. Thus, when the heterologous region comprises a gene, the gene will usually be flanked by DNA that does not flank the genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

In reference to mutant plants, the terms “null mutant” or “loss-of-function mutant” are used to designate an organism or genomic DNA sequence with a mutation that causes a gene product to be non-functional or largely absent. Such mutations may occur in the coding and/or regulatory regions of the gene, and may be changes of individual residues, or insertions or deletions of regions of nucleic acids. These mutations may also occur in the coding and/or regulatory regions of other genes which may regulate or control a gene and/or encoded protein, so as to cause the protein to be non-functional or largely absent.

“Grain,” “seed,” or “bean,” refers to a flowering plant's unit of reproduction, capable of developing into another such plant. As used herein, especially with respect to coffee plants, the terms are used synonymously and interchangeably.

An “enzyme” is a protein that has enzymatic activity.

“Galactomannan precursor synthesis enzyme” and “galactomannan precursor synthesis gene” refers to a protein, or enzyme, and the gene that encodes the same, involved in the synthesis of precursor molecules needed for synthesis of galactomannan polymers. Galactomannan precursor synthesis enzymes include UDP-glucose pyrophosphorylase (UGPP), UDP-glucose 4-epimerase (UGE), phosphomannomutase (PMM) and GDP-mannose pyrophosphorylase (GMPP). Likewise, galactomannan precursor synthesis genes include genes that encode UGPP, UGE, PMM and GMPP.

As used herein, the term “plant” includes reference to whole plants, plant organs (e.g., leaves, stems, branches, shoots, roots), seeds, pollen, plant cells, plant cell organelles, and progeny thereof, including fertile progeny. Parts of transgenic plants are to be understood within the scope of the invention to comprise, for example, plant cells, protoplasts, tissues, callus, embryos as well as flowers, stems, branches, seeds, pollen, fruits, leaves, or roots originating in transgenic plants or their progeny.

Ranges are used herein as shorthand to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “an enzyme,” “a plant”, or “a method”, or “a disease” includes a plurality of such “enzymes,” “plants” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of”.

The methods and compositions and other advances disclosed herein are not limited to particular methodologies, protocols, and reagents because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### Description

The galactomannans are an important group of polysaccharides found in the green coffee grain. It is known that this particular coffee polymer is difficult to solubilize. Accordingly, it has been an object of certain research efforts to find ways to reduce the amount of galactomannan in coffee grain, thereby achieving higher solubility of roasted coffee at lower temperatures. Another research object has been to alter the solubility of galactomannan in coffee grain, so that it is easier to extract at lower temperatures, even if abundantly present. Heretofore, such efforts have focused on altering the amount or activity of enzymes involved in galactomannan synthesis and degradation, using galactosyltransferase (GMGTase), mannan synthase (ManS) and mannanases. Other, more global efforts have undertaken to determine the realationship between numerous metabolic pathways, using Coffea Arabica as a case study, but have not focused in particular on the galactomannan pathway (Joet et al., April 2009, New Phytol. 182(1), 146-162).

The present invention springs in part from the inventors' insight that the galactomannan content or structure within coffee grain may also be modulated by altering the availability of the substrates or upstream intermediates for the synthetic enzymes (GMGTase and ManS), i.e., mannose 1-phosphate, GDP-mannose, UDP-glucose and UDP-galactose. Further, the inventors have appreciated that this can be accomplished on a biological level by modulating the amount or activity of the enzymes involved in the formation of these precursors, which include: (1) UDP-glucose pyrophosphorylase (UGPP), catalyzing the conversion of glucose-1-phosphate to UDP-glucose; (2) UDP-glucose 4-epimerase (UGE), catalyzing the conversion of UDP-glucose to UDP-galactose; (3) phosphomannomutase (PMM), catalyzing the conversion of mannose-6-phosphate to mannose-1-phosphate; and (4) GDP-mannose pyrophosphorylase (GMPP), catalyzing the conversion of mannose-1-phosphate to GDP-mannose.

As described in detail below, the inventors have isolated Coffea canephora cDNA for these genes and determined their expression during the development of coffee cherries and in several other coffee tissues. Methods for utilizing these genes and their encoded enzymes to modulate galactomannan precursors synthesis, aimed at modulating the galactomannan level and or solubility in coffee, have also been devised.

Polynucleotides and Polypeptides:

One aspect of the present invention features nucleic acid molecules from coffee that encode enzymes involved in synthesis of galactomannan precursors. These include UDP-glucose pyrophosphorylase (UGPP), GDP-mannose pyrophosphorylase (GMPP), phosphomannomutase (PMM), and UDP-glucose 4-epimerase (UGE), and are sometimes referred to collectively as “galactomannan precursor synthesis enzymes.” A cDNA encoding a complete UGPP from Coffea canephora is set forth herein as SEQ ID NO: 1, and is referred to as CcUGPP. A cDNA encoding a complete GMPP from C. canephora is set forth herein as SEQ ID NO:2, and is referred to as CcGMPP. A cDNA encoding a complete PMM from C. canephora is set forth herein as SEQ ID NO: 3, and is referred to as CcPMM. Two cDNAs encoding complete UGEs from C. canephora are set forth herein as SEQ ID NO:4 and SEQ ID NO:5, and are referred to as CcUGE1 and CcUGE5, respectively.

Another aspect of the invention features the proteins produced by expression of these nucleic acid molecules. The deduced amino acid sequences of the CcUGPP protein produced by translation of SEQ ID NO:1 is set forth herein as SEQ ID NO:6. The deduced amino acid sequence of the CcGMPP protein produced by translation of SEQ ID NO:2 is set forth herein as SEQ ID NO:7. The deduced amino acid sequences of the CcPMM protein produced by translation of SEQ ID NO:3 is set forth herein as SEQ ID NO:8. The deduced amino acid sequences of the CcUGE1 and CcUGE5 proteins produced by translation of SEQ ID NO:4 and SEQ ID NO:5 are set forth herein as SEQ ID NO:9 and SEQ ID NO:10, respectively.

Although galactomannan precursor synthesis polynucleotides and enzymes from Coffea canephora are exemplified herein, this invention is intended to encompass nucleic acids and encoded proteins from other Coffea species that are sufficiently similar to be used interchangeably with the C. canephora polynucleotides and proteins for the purposes described below. Accordingly, when the galactomannan precursor synthesis enzymes “UDP-glucose pyrophosphorylase” (“UGPP”), “GDP-mannose pyrophosphorylase” (“GMPP”), “phosphomannomutase” (“PMM”), and “UDP-glucose 4-epimerase” (“UGE”) are referred to herein, these terms are intended to encompass all Coffea UGPPs, GMPPs, PMMs and UGEs having the general physical, biochemical and functional features described herein, and polynucleotides encoding them, unless specifically stated otherwise.

Considered in terms of their sequences, UGPP, GMPP, PMM and UGE polynucleotides of the invention include allelic variants and natural mutants of SEQ ID NOS: 1-5, which are likely to be found in different varieties of C. canephora and Coffea arabica, as well as variants, natural mutants and homologs of SEQ ID NOs: 1-5 that are likely to be found in different coffee species, including but not limited to C. arabica. In particular embodiments, variants, mutants and homologs from C. arabica are employed. Because such variants and homologs are expected to possess certain differences in nucleotide and amino acid sequence, suitable galatomannan precursor synthesis polypeptides include those having at least about 80%, or 81%, or 82%, or 83%, or 84%, or 85%, or 86%, or 87%, or 88%, or 89%, or 90%, or 91%, or 92%, or 93%, or 94%, or 95%, or 96%, or 97%, or 98% or 99% identity with the polypeptide of SEQ ID NOS: 6-10, respectively. Because of the natural sequence variation likely to exist among these enzymes, and the genes encoding them in different coffee varieties and species, one skilled in the art would expect to find this level of variation, while still maintaining the unique properties of the polypeptides and polynucleotides of the present invention. Such an expectation is due in part to the degeneracy of the genetic code, as well as to the known evolutionary success of conservative amino acid sequence variations, which do not appreciably alter the nature of the encoded protein.

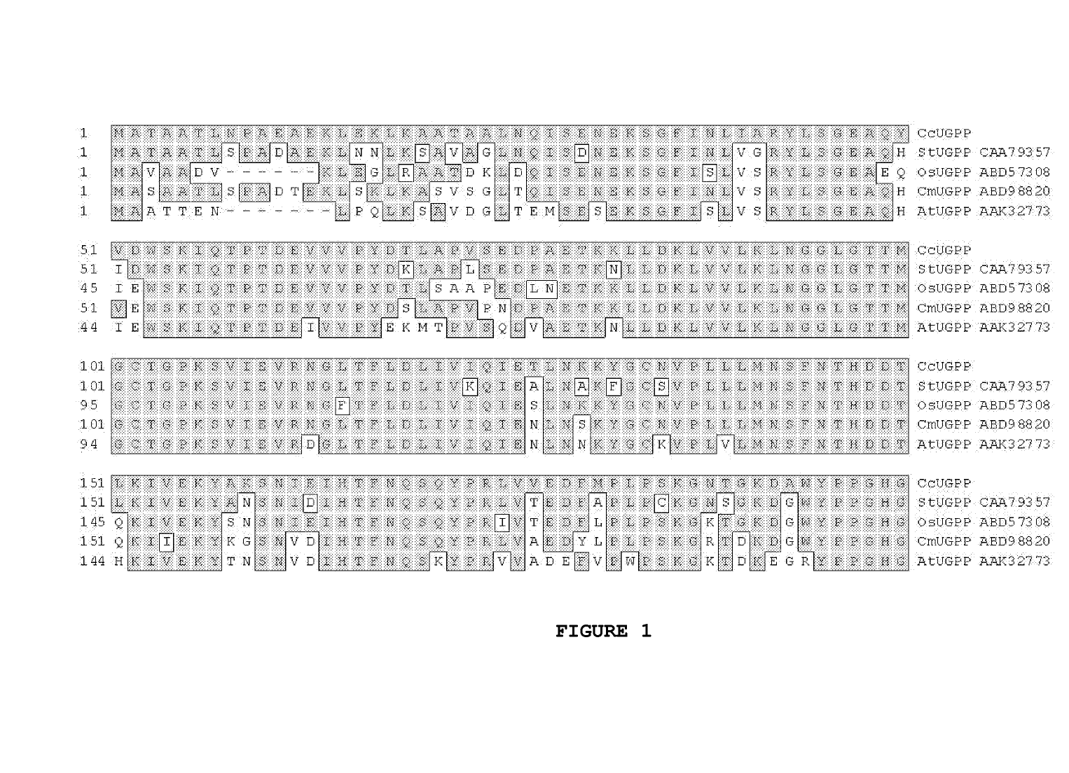

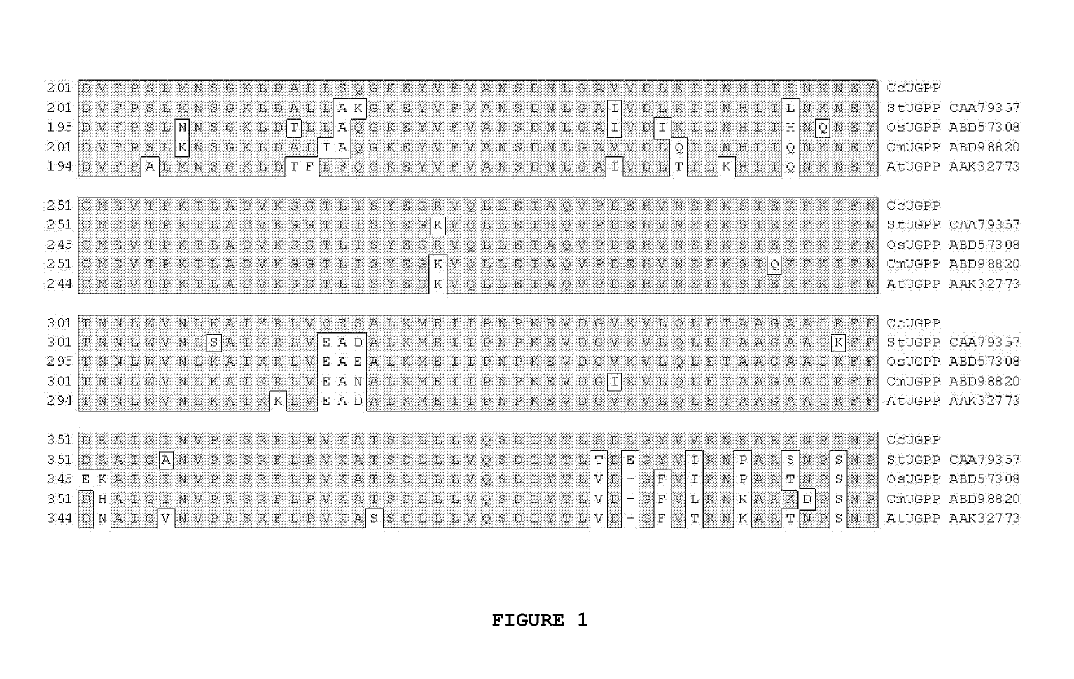

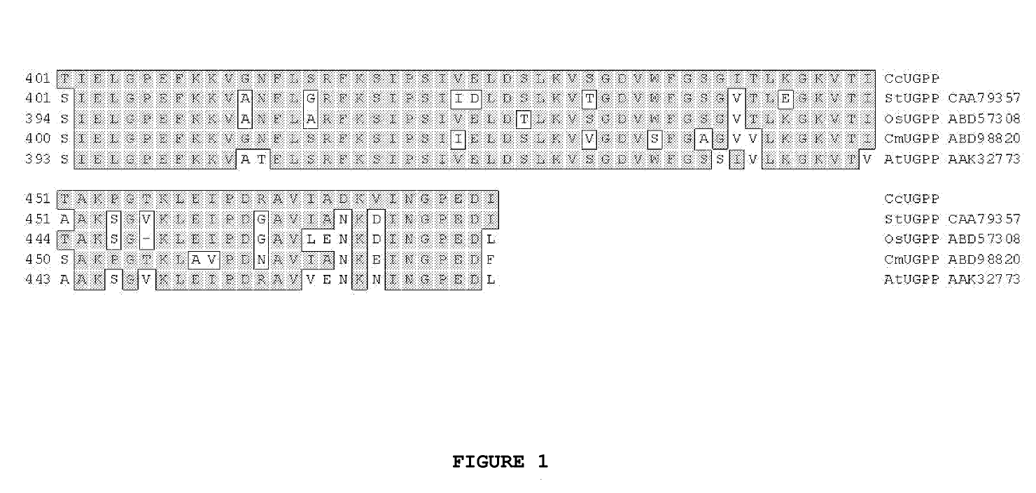

The C. canephora galactomannan precursor enzymes can be further distinguished from orthologs from other species by regions of the proteins having non-conserved sequences. Unique or non-conserved sequences for each of CcUGPP, CcGMPP, CcPMM, CcUGE1 and CcUGE5 are set forth below (single residues are set forth as such; contiguous sequences of two or more sequences are noted with a hyphen between the two residues; e.g., “1-8” means contiguous residues 1 through 8, inclusive).

| | SEQ | |
|---|---|---|
| Enzyme | ID NO: | Position (taken from numbering in FIGS. 1-4) |
| CcUGPP: | 6 | 1-17; 68-78; 127-132; 158-163; 177-181; 188-194; |
| | | 214-218; 316-318; 383-398; 440-445; and 466-470 |
| CcGMPP: | 7 | 44; 66; 74; 66-74; 98; 100; 98-100; 118; 150; |
| | | 153; 185; 188; 207; 241-242; 249; and 258 |
| CcPMM: | 8 | 1-9; 24-31; 24-35; 41; 63; 77; 79; 197; 217; |
| | | 238; 239; 241; 242; 238-246; and 246 |
| CcUGE1: | 9 | 1-8; 28-29; 34; 39; 28-44; 44; 48; 28-48; 54; |
| | | 28-65; 65; 73; 77; 78; 82; 83; 65-85; 85; |
| | | 101-104; 101; 102; 104; 101-119; 112; 119; |
| | | 140-151; 151; 170; 170-176; 197-198; 214-215; |
| | | 261-265; 261-268; 268; 288; 288-289; 294; |
| | | 328-351; 328; 331; 334; 342; 348; 349 |
| CcUGE5: | 10 | 1-6; 43-45; 56-74; 98-104; 164-167; 219-226; |
| | | 293-296; 305; 309; 311; 323-349 |

Polynucleotides and Polypeptides:

Nucleic acid molecules of the invention may be prepared by two general methods: (1) they may be synthesized from appropriate nucleotide triphosphates, or (2) they may be isolated from biological sources. Both methods utilize protocols well known in the art.

The availability of nucleotide sequence information, such as the cDNA having SEQ ID NOS: 1-5, enables preparation of isolated nucleic acid molecules by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC).

Nucleic acids having the appropriate level of sequence homology with part or all of the coding and/or regulatory regions of galactomannan precursor synthesis polynucleotides may be identified by using hybridization and washing conditions of appropriate stringency. It will be appreciated by those skilled in the art that the aforementioned strategy, when applied to genomic sequences, will, in addition to enabling isolation of enzyme coding sequences, also enable isolation of promoters and other gene regulatory sequences associated with galactomannan precursor synthesis genes, even though the regulatory sequences themselves may not share sufficient homology to enable suitable hybridization.

As a typical illustration, hybridizations may be performed using a hybridization solution comprising: 5×SSC, 5×Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 2×SSC and 0.1% SDS; (4) 2 hours at 45-55° C. in 2×SSC and 0.1% SDS, changing the solution every 30 minutes.

One common formula for calculating the stringency conditions required to achieve hybridization between nucleic acid molecules of a specified sequence homology (Sambrook et al., 1989, Molecular Cloning, A Laboratory Manual (2^{nd} Ed.); Cold Spring Harbor):

Tm=81.5° C.+16.6 Log [Na+]+0.41 (% G+C)−0.63 (% formamide)−600/#bp in duplex

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C. In one embodiment, the hybridization is at 37° C. and the final wash is at 42° C.; in another embodiment the hybridization is at 42° C. and the final wash is at 50° C.; and in yet another embodiment the hybridization is at 42° C. and final wash is at 65° C., with the above hybridization and wash solutions. Conditions of high stringency include hybridization at 42° C. in the above hybridization solution and a final wash at 65° C. in 0.1×SSC and 0.1% SDS for 10 minutes.

Nucleic acids may be maintained as DNA in any convenient cloning vector. In a preferred embodiment, clones are maintained in plasmid cloning/expression vector, such as pGEM-T (Promega Biotech, Madison, Wis.), pBluescript (Stratagene, La Jolla, Calif.), pCR4-TOPO (Invitrogen, Carlsbad, Calif.) or pET28a+(Novagen, Madison, Wis.), all of which can be propagated in a suitable E. coli host cell.

Nucleic acid molecules of the invention include cDNA, genomic DNA, RNA, and fragments thereof which may be single-, double-, or even triple-stranded. Thus, this invention provides oligonucleotides (sense or antisense strands of DNA or RNA) having sequences capable of hybridizing with at least one sequence of a nucleic acid molecule of the present invention. Such oligonucleotides are useful as probes for detecting galactomannan precursor synthesis genes or mRNA in test samples of plant tissue, e.g., by PCR amplification, or for the positive or negative regulation of expression of galactomannan precursor synthesis enzymes at or before translation of the mRNA into proteins. Methods in which oligonucleotides or polynucleotides may be utilized as probes for such assays include, but are not limited to: (1) in situ hybridization; (2) Southern hybridization (3) northern hybridization; and (4) assorted amplification reactions such as polymerase chain reactions (PCR, including RT-PCR) and ligase chain reaction (LCR).

Optionally, oligonucleotides may be constructed to comprise regions of the galactomannan precursor enzyme-encoding polynucleotides that are unique to those polynucleotides, i.e, that are more likely to hybridize with Coffea polynucleotides than to orthologs from other species. Suitable regions for targeting in this manner include regions encoding the unique or non-conserved regions for each of the encoded proteins, as set forth above.

The oligonucleotides having sequences capable of hybridizing with at least one sequence of a nucleic acid molecule of the present invention include antisense oligonucleotides. The antisense oligonucleotides are targeted to specific regions of the mRNA that are critical for translation may be utilized. The use of antisense molecules to decrease expression levels of a pre-determined gene is known in the art. Antisense molecules may be provided in situ by transforming plant cells with a DNA construct which, upon transcription, produces the antisense RNA sequences. Such constructs can be designed to produce full-length or partial antisense sequences. This gene silencing effect can be enhanced by transgenically over-producing both sense and antisense RNA of the gene coding sequence so that a high amount of dsRNA is produced. In this regard, dsRNA containing sequences that correspond to part or all of at least one intron have been found particularly effective. In one embodiment, part or all of the appropriate antisense strand is expressed by a transgene. In another embodiment, genes may be silenced by use of small interfering RNA (siRNA) or micro-RNA (miRNA) using commercially available materials and methods (e.g., Invitrogen, Inc., Carlsbad Calif.).

Polypeptides may be prepared in a variety of ways, according to known methods. If produced in situ the polypeptides may be purified from appropriate sources, e.g., seeds, pericarps, or other plant parts. Alternatively, the availability of nucleic acid molecules encoding the polypeptides enables production of the proteins using in vitro expression methods known in the art.

For instance, quantities of polypeptides may be produced by expression in a suitable procaryotic or eucaryotic system. For example, part or all of a DNA molecule, such as the cDNA having any of SEQ ID NOs: 1-5, may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

The polypeptides produced by gene expression in a recombinant procaryotic or eucaryotic system may be purified according to methods known in the art. In a preferred embodiment, a commercially available expression/secretion system can be used, whereby the recombinant protein is expressed and thereafter secreted from the host cell, and, thereafter, purified from the surrounding medium. An alternative approach involves purifying the recombinant protein by affinity separation, e.g., via immunological interaction with antibodies that bind specifically to the recombinant protein. The polypeptides of the invention, prepared by the aforementioned methods, may be analyzed according to standard procedures.

Polypeptides purified from coffee or recombinantly produced, may be used to generate polyclonal or monoclonal antibodies, antibody fragments or derivatives as defined herein, according to known methods. Optionally, antibodies made against synthetic peptides corresponding to nonconserved regions of the respective proteins can be generated.

Vectors, Kits and Transgenic Organisms:

Also featured in accordance with the present invention are vectors and kits for producing transgenic host cells that contain galactomannan precursor synthesis polynucleotides, oligonucleotides, variants thereof in a sense or antisense orientation, siRNA, miRNA or reporter genes and other constructs under control of appropriate promoters and other regulatory sequences. Suitable host cells include, but are not limited to, plant cells, bacterial cells, yeast and other fungal cells, insect cells and mammalian cells. Vectors for transforming a wide variety of these host cells are well known to those of skill in the art. They include, but are not limited to, plasmids, cosmids, baculoviruses, bacmids, bacterial artificial chromosomes (BACs), yeast artificial chromosomes (YACs), as well as other bacterial, yeast and viral vectors. Typically, kits for producing transgenic host cells will contain one or more appropriate vectors and instructions for producing the transgenic cells using the vector. Kits may further include one or more additional components, such as culture media for culturing the cells, reagents for performing transformation of the cells and reagents for testing the transgenic cells for gene expression, to name a few.

The present invention includes transgenic plants comprising one or more copies of a galactomannan precursor synthesis polynucleotide, or nucleic acid sequences, such as antisense, siRNA or miRNA, that inhibit the production or function of one or more of a plant's endogenous galactomannan precursor synthesis enzymes. This is accomplished by transforming plant cells with a transgene that comprises part or all of a galactomannan precursor synthesis enzyme coding sequence, or mutant, antisense or variant thereof, including RNA, siRNA or miRNA, controlled by either native or recombinant regulatory sequences, as described below. Transgenic coffee species include, without limitation, C. abeokutae, C. arabica, C. arnoldiana, C. aruwemiensis, C. bengalensis, C. canephora, C. congensis C. Dewevrei, C. excelsa, C. eugenioides, C. heterocalyx, C. kapakata, C. khasiana, C. liberica, C. moloundou, C. rasemosa, C. salvatrix, C. sessiflora, C. stenophylla, C. travencorensis, C. wightiana and C. zanguebariae. Plants of any species are also included in the invention, since the methods described below may be of particular advantage in modulating galactomannan content in other species. Such species include, but are not limited to, tobacco, Arabidopsis and other “laboratory-friendly” species, cereal crops such as maize, wheat, rice, soybean barley, rye, oats, sorghum, alfalfa, clover and the like, oil-producing plants such as canola, safflower, sunflower, peanut, cacao and the like, vegetable crops such as tomato tomatillo, potato, pepper, eggplant, sugar beet, carrot, cucumber, lettuce, pea and the like, horticultural plants such as aster, begonia, chrysanthemum, delphinium, petunia, zinnia, lawn and turfgrasses and the like.

Transgenic plants can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors or other plant viral vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods are well known in the art.

The method of transformation depends upon the plant to be transformed. Agrobacterium vectors are often used to transform dicot species. Agrobacterium binary vectors include, but are not limited to, BIN19 and derivatives thereof, the pBI vector series, and binary vectors pGA482, pGA492, pLH7000 (GenBank Accession AY234330) and any suitable one of the pCAMBIA vectors (derived from the pPZP vectors constructed by Hajdukiewicz et al., 1994, Plant Mol Biol 25, 989-994, available from CAMBIA, GPO Box 3200, Canberra ACT 2601, Australia or via the worldwide web at CAMBIA.org). For transformation of monocot species, biolistic bombardment with particles coated with transforming DNA and silicon fibers coated with transforming DNA are often useful for nuclear transformation. Alternatively, Agrobacterium “superbinary” vectors have been used successfully for the transformation of rice, maize and various other monocot species.

DNA constructs for transforming a selected plant comprise a coding sequence of interest operably linked to appropriate 5′ (e.g., promoters and translational regulatory sequences) and 3′ regulatory sequences (e.g., terminators). In one embodiment, a galactomannan precursor synthesis coding sequence under control of its own 5′ and 3′ regulatory elements can be utilized.

In other embodiments, galactomannan precursor synthesis coding and regulatory sequences are swapped to alter the polysaccharide profile of the transformed plant.

In an alternative embodiment, the coding region of the gene is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase and octopine synthase promoters. In other embodiments, a strong monocot promoter is used, for example, the maize ubiquitin promoter, the rice actin promoter or the rice tubulin promoter (Jeon et al., 2000, Plant Physiology 123, 1005-14).

Transgenic plants expressing galactomannan precursor synthesis enzyme coding sequences under an inducible promoter are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene) to name a few.

Tissue specific and development-specific promoters are also contemplated for use in the present invention. Non-limiting examples of seed-specific promoters include Cim1 (cytokinin-induced message), cZ19B1 (maize 19 kDa zein), milps (myo-inositol-1-phosphate synthase), and celA (cellulose synthase) (U.S. Pat. No. 6,225,529), bean beta-phaseolin, napin, beta-conglycinin, soybean lectin, cruciferin, maize 15 kDa zein, 22 kDa zein, 27 kDa zein, g-zein, waxy, shrunken 1, shrunken 2, and globulin 1, soybean 11S legumin, and C. canephora 11S seed storage protein. See also WO 00/12733, where seed-preferred promoters from end1 and end2 genes are disclosed. Other Coffea seed specific promoters may also be utilized, including but not limited to the oleosin gene promoter described in WO 2007/005928, the dehydrin gene promoter described in WO 2007/005980, and the 9-cis-epoxycarotenoid dioxygenase gene promoter described in WO 2007/028115. Examples of other tissue-specific promoters include, but are not limited to: the ribulose bisphosphate carboxylase (RuBisCo) small subunit gene promoters (e.g., U.S. Pat. No. 7,153,953 to Marraccini et al.) or chlorophyll a/b binding protein (CAB) gene promoters for expression in photosynthetic tissue; and the root-specific glutamine synthetase gene promoters where expression in roots is desired.

The coding region is also operably linked to an appropriate 3′ regulatory sequence. In embodiments where the native 3′ regulatory sequence is not used, the nopaline synthetase polyadenylation region may be used. Other useful 3′ regulatory regions include, but are not limited to the octopine synthase polyadenylation region.

The selected coding region, under control of appropriate regulatory elements, is operably linked to a drug resistance marker, such as kanamycin resistance. Other useful selectable marker systems include genes that confer antibiotic or herbicide resistances (e.g., resistance to hygromycin, sulfonylurea, phosphinothricin, or glyphosate) or genes conferring selective growth (e.g., phosphomannose isomerase, enabling growth of plant cells on mannose). Selectable marker genes include, without limitation, genes encoding antibiotic resistance, such as those encoding neomycin phosphotransferase II (NEO), dihydrofolate reductase (DHFR) and hygromycin phosphotransferase (HPT), as well as genes that confer resistance to herbicidal compounds, such as glyphosate-resistant EPSPS and/or glyphosate oxidoreducatase (GOX), Bromoxynil nitrilase (BXN) for resistance to bromoxynil, AHAS genes for resistance to imidazolinones, sulfonylurea resistance genes, and 2,4-dichlorophenoxyacetate (2,4-D) resistance genes.

In certain embodiments, promoters and other expression regulatory sequences encompassed by the present invention are operably linked to reporter genes. Reporter genes contemplated for use in the invention include, but are not limited to, genes encoding green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), β-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). As with many of the standard procedures associated with the practice of the invention, skilled artisans will be aware of additional sequences that can serve the function of a marker or reporter.

Additional sequence modifications are known in the art to enhance gene expression in a cellular host. These modifications include elimination of sequences encoding superfluous polyadenylation signals, exon-intron splice site signals, transposon-like repeats, and other such well-characterized sequences that may be deleterious to gene expression. Alternatively, if necessary, the G/C content of the coding sequence may be adjusted to levels average for a given coffee plant cell host, as calculated by reference to known genes expressed in a coffee plant cell. Also, when possible, the coding sequence is modified to avoid predicted hairpin secondary mRNA structures. Another alternative to enhance gene expression is to use 5′ leader sequences. Translation leader sequences are well known in the art, and include the cis-acting derivative (omega') of the 5′ leader sequence (omega) of the tobacco mosaic virus, the 5′ leader sequences from brome mosaic virus, alfalfa mosaic virus, and turnip yellow mosaic virus.

Plants are transformed and thereafter screened for one or more properties, including the presence of the transgene product, the transgene-encoding mRNA, or an altered phenotype associated with expression of the transgene or the expression of a sequence designed to decrease expression an endogenous gene, e.g., antisense, siRNA or miRNA. It should be recognized that the amount of expression, as well as the tissue- and temporal-specific pattern of expression of the transgenes in transformed plants can vary depending on the position of their insertion into the nuclear genome. Such positional effects are well known in the art. For this reason, several nuclear transformants should be regenerated and tested for expression of the transgene.

Methods:

The nucleic acids and polypeptides of the present invention can be used in any one of a number of methods whereby production or activity of one or more of the galactomannan precursor synthesis enzymes in coffee plants can be modulated to affect various phenotypic traits, e.g., for improvement in the production qualities of the beans. For instance, a decrease in galactomannan content, or an alteration of galactomannan structure, is expected to greatly improve recovery of solids in the process of making instant coffee. An increase in galactomannan content may be desirable for other parts of the plant, or for other plant species as well.

Improvement of coffee grain galactomannan content or structure, or other characteristics, can be obtained by (1) classical breeding or (2) genetic engineering techniques, and by combining these two approaches. Both approaches have been considerably improved by the isolation and characterization of polynucleotides encoding the galactomannan precursor synthesis enzymes UGPP, GMPP, PMM and/or UGE in coffee, in accordance with the present invention. For example, the UGPP-, GMPP-, PMM- and/or UGE-encoding genes may be genetically mapped and Quantitative Trait Loci (QTL) involved in galactomannan content or structure can be identified. It would then be possible to determine if such QTL correlate with the position of the UGPP, GMPP, PMM or UGE related genes. Alleles (haplotypes), for genes affecting levels of galactomannan precursors may also be identified and examined to determine if the presence of specific haplotypes are strongly correlated with galactomannan precursor synthesis. These markers can be used to advantage in marker assisted breeding programs.

Another advantage of isolating polynucleotides involved in galactomannan precursor synthesis has been demonstrated herein by the present inventors. This is to generate expression data for these genes during coffee bean maturation in varieties with high and low galactomannan or galactomannan precursor levels. The information is used to direct the choice of genes to use in genetic manipulation aimed at generating novel transgenic coffee plants that have increased or decreased galactomannan levels in the mature bean.

In one aspect, the present invention features methods to alter the galactomannan profile in a plant, preferably coffee, comprising increasing or decreasing an amount or activity of one or more galactomannan precursor synthesis enzymes in the plant. Specific embodiments of the present invention provide methods for increasing or decreasing production of UGPP, GMPP, PMM and/or UGE.

In one embodiment, coffee plants can be transformed with one or more of a UGPP, GMPP, PMM and/or UGE-encoding polynucleotide, such as a cDNA comprising SEQ ID NOs: 1-5, for the purpose of over-producing one or more of these enzymes, respectively, in various tissues of coffee. In one embodiment, coffee plants are engineered for a general increase in UGPP, GMPP, PMM and/or UGE production, e.g., through the use of a promoter such as the RuBisCo small subunit (SSU) promoter or the CaMV35S promoter functionally linked to the coding sequence. In some embodiments, the modification of coffee plants can be engineered to increase two, three, or all of UGPP, GMPP, PMM or UGE.

Transgenic plants comprising one or more of the aforementioned UGPP, GMPP, PMM or UGE coding sequences may also contain coding sequences for the enzymes involved directly in galactomannan synthesis, i.e., mannan synthase and galactomannan galactosyltransferases, such as described in WO 2007/047675. They may also optionally contain RNAi-encoding sequences (as described below) targeted to RNA encoding the galactomannan degrading enzymes. Combinations of one or more of these transgenes should result in effective up-regulation of galactomannan synthesis at several levels in the biosynthetic pathway, with optional down-regulation of galactomannan degradative enzymes.

One situation that could arise in an effort to build pools of galactomannan precursors is that such precursors could be siphoned off into other biochemical pathways and therefore not be available for galactomannan synthesis. One way to circumvent such a situation would be to utilize one or more galactomannan precursor synthesis genes from a different plant species.

This would be expected to circumvent such siphoning, and avoid issues that could arise from species-specific translational and post-translational inhibition. Such phenomena have been observed in sucrose metabolism in plants (Privat, et al., 2008, New Phytol. 178, 781-797).

In another embodiment designed to limit over-production of the galatomannan precursor enzyme(s) only to a sink organ of interest, i.e., the grain, a grain-specific promoter may be utilized, particularly one of the Coffea grain-specific promoters described above. These promoters are also of use to direct expression of polynucleotides intended to down-regulate expression of a target gene, as described below.

Plants exhibiting altered galactomannan or galactomannan precursor profiles can be screened for naturally-occurring variants of UGPP, GMPP, PMM and/or UGE, e.g., by measuring formation of galactomannan precursors and, optionally, galactomannan, or by measuring amount or activity of the various enzymes. For instance, loss-of-function (null) mutant plants may be created or selected from populations of plant mutants currently available. It will also be appreciated by those of skill in the art that mutant plant populations may also be screened for mutants that under or over-express a particular polysaccharide metabolizing enzyme, such as a galactomannan precursor synthesis enzyme, utilizing one or more of the methods described herein. Mutant populations can be made by chemical mutagenesis, radiation mutagenesis, and transposon or T-DNA insertions, or targeting induced local lesions in genomes (TILLING, see, e.g., Henikoff et al., 2004, Plant Physiol. 135, 630-636; Gilchrist & Haughn, 2005, Curr. Opin. Plant Biol. 8, 211-215). The methods to make mutant populations are well known in the art.

The nucleic acids of the invention can be used to identify mutant forms of galactomannan precursor synthesis enzymes in various plant species. In species such as maize or Arabidopsis, where transposon insertion lines are available, oligonucleotide primers can be designed to screen lines for insertions in the galactomannan precursor synthesis genes. Through breeding, a plant line may then be developed that is heterozygous or homozygous for the interrupted gene. Heterozyocity may be more useful than homozygocity in some embodiments, inasmuch as complete ablation of a biosynthetic enzyme could be too detrimental for plants to survive, whereas partial ablation may yield a more desirable result.

Another embodiment of the present invention involves decreasing galactomannan in coffee grain by decreasing the amount or activity of one or more of UGPP, GMPP, PMM and/or UGE in the grain. This may be accomplished in a variety of ways.

In one embodiment, a plant may be engineered to display a phenotype similar to that seen in null mutants created by mutagenic techniques. A transgenic null mutant can be created by expressing a mutant form of UGPP, GMPP, PMM and/or UGE to create a “dominant negative effect.” While not limiting the invention to any one mechanism, this mutant protein will compete with wild-type protein for interacting proteins or other cellular factors. Examples of this type of “dominant negative” effect are well known for both insect and vertebrate systems.

Another kind of transgenic null mutant can be created by inhibiting the translation of UGPP, GMPP, PMM and/or UGE-encoding mRNA by “post-transcriptional gene silencing.” These techniques may be used to down-regulate the enzyme(s) in a plant grain, thereby decreasing the amount of galatomannan precursors available for galactomannan synthesis. For instance, a galactomannan precursor synthesis polynucleotide, or a fragment thereof, may be utilized to control the production of the encoded protein. Full-length antisense molecules can be used for this purpose. Alternatively, antisense oligonucleotides targeted to specific regions of the mRNA that are critical for translation may be utilized. The use of antisense molecules to decrease expression levels of a pre-determined gene is known in the art. Antisense molecules may be provided in situ by transforming plant cells with a DNA construct which, upon transcription, produces the antisense RNA sequences. Such constructs can be designed to produce full-length or partial antisense sequences. This gene silencing effect can be enhanced by transgenically over-producing both sense and antisense RNA of the gene coding sequence so that a high amount of dsRNA is produced (for example see Waterhouse et al., 1998, Proc Natl Acad Sci USA 95, 13959-13964). In this regard, dsRNA containing sequences that correspond to part or all of at least one intron have been found particularly effective. In one embodiment, part or all of a UGPP, GMPP, PMM and/or UGE-encoding antisense strand is expressed by a transgene.

In another embodiment, galactomannan precursor synthesis genes may be silenced through the use of a variety of other post-transcriptional gene silencing (RNA silencing) techniques that are currently available for plant systems. RNA silencing involves the processing of double-stranded RNA (dsRNA) into small 21-28 nucleotide fragments by an RNase H-based enzyme (“Dicer” or “Dicer-like”). The cleavage products, which are siRNA (small interfering RNA) or miRNA (micro-RNA) are incorporated into protein effector complexes that regulate gene expression in a sequence-specific manner (for reviews of RNA silencing in plants, see Horiguchi, 2004, Differentiation 72, 65-73; Baulcombe, 2004, Nature 431, 356-363; Herr, 2004, Biochem. Soc. Trans. 32, 946-951). siRNA is perfectly base paird to its target, and is believed to reduce expression by cleaving the target RNA. By comparison, miRNAs regulate gene expression by forming imperfectly base-paired duplexes with target mRNAs, most often within the 3′ non-coding region of the message. Generally, miRNAs inhibit translation of target mRNAs, although in some cases they might also reduce the half life and therefore the level of targeted mRNAs.

Small interfering RNAs or micro-RNAs may be chemically synthesized or transcribed and amplified in vitro, and then delivered to the cells. Delivery may be through microinjection, chemical transfection, electroporation or cationic liposome-mediated transfection, or any other means available in the art, which will be appreciated by the skilled artisan. Alternatively, the miRNA or siRNA may be expressed intracellularly by inserting DNA templates for miRNA or siRNA into the cells of interest, for example, by means of a plasmid, and may be specifically targeted to select cells. Small interfering RNAs have been successfully introduced into plants.

A preferred method of RNA silencing in the present invention is the use of short hairpin RNAs (shRNA). A vector containing a DNA sequence encoding for a particular desired siRNA sequence is delivered into a target cell by any common means. Once in the cell, the DNA sequence is continuously transcribed into RNA molecules that loop back on themselves and form hairpin structures through intramolecular base pairing. These hairpin structures, once processed by the cell, are equivalent to siRNA molecules and are used by the cell to mediate RNA silencing of the desired protein. Various constructs of particular utility for RNA silencing in plants are described by Horiguchi, 2004, supra. Typically, such a construct comprises a promoter, a sequence of the target gene to be silenced in the “sense” orientation, a spacer, the antisense of the target gene sequence, and a terminator.

Yet another type of synthetic null mutant can also be created by the technique of “co-suppression” (Vaucheret et al., 1998, Plant J. 16, 651-659). Plant cells are transformed with a copy of the endogenous gene targeted for repression. In many cases, this results in the complete repression of the native gene as well as the transgene. In one embodiment, a galactomannan precursor synthesis gene from the plant species of interest is isolated and used to transform cells of that same species.

Any of the aforementioned techniques may be applied not only to UGPP, GMPP, PMM or UGE coding sequences, but may also include inhibiting expression of coding sequences for the enzymes involved directly in galactomannan synthesis, i.e., mannan synthases and galactomannan galactosyltransferases, such as those described in WO 2007/047675. The techniques may optionally be combined with over-expression of one or more mannanases, to accelerate galactomannan degradation in a selected tissue. Combinations of one or more of these transgenes should result in effective down-regulation of galactomannan synthesis at several levels in the biosynthetic pathway, with optional up-regulation of galactomannan degradative enzymes.

An important consideration in applying the aforementioned translation inhibitory techniques is the timing of such inhibition. It is advantageous to select one or more of the galactomannan precursor synthesis genes that is expressed in the coffee seed at the right moment, then design the RNAi construct to lower expression of that gene. Gene control should not only be development-specific, but also tissue specific, e.g., grain specific, optionally sub-specific to a selected part of the grain. The gene expression data for CcUGPP, CcGMPP, CcPMM and CcUGE set forth in Example 4 are useful for the purpose of making selections based on such parameters. For instance, the grain expression data for the four genes indicates that the two more “upstream” genes, UGPP and PMM, are expressed in a relatively uniform manner over the stages of grain development, while the genes downstream, UGE and particularly GMPP, showed somewhat more developmentally related profiles (notably, GMPP expression was observed to decrease in the latest stage of development), indicating their expression could more closely reflect the actual needs of the galactomannan synthesis and other UDP-galactose and GDP-mannose reactions. Thus, one embodiment of the invention features selective inhibition of GMPP and/or UGE in coffee grain at the developmental stage in which their expression is higher. The data presented herein also suggest that different alleles of UGE have different effects in different coffee varieties. Accordingly, another embodiment features selective manipulation of UGE1 or UGE5, separately or together, depending on variety. In another embodiment, UGPP may be down-regulated and UGE1 and/or UGE5 up-regulated at the time in development when GMPP expression is highest. Such manipulation could direct sucrose toward UDP-galactose, thereby down-regulating GMPP. Such manipulations would benefit by optimization of the promoters used, including the coffee promoters described above.

Mutant or transgenic plants produced by any of the foregoing methods are also featured in accordance with the present invention. Preferably, the plants are fertile, thereby being useful for breeding purposes. Thus, mutant or plants that exhibit one or more of the aforementioned desirable phenotypes can be used for plant breeding, or directly in agricultural or horticultural applications. Plants containing one transgene or a specified mutation may also be crossed with plants containing a complementary transgene or genotype in order to produce plants with enhanced or combined phenotypes.

The following examples are provided to describe the invention in greater detail. The examples are for illustrative purposes, and are not intended to limit the invention.

### Example 1

### Materials and Methods for Subsequent Examples

Plant material. To follow the gene expression by Q-PCR, one Coffea arabica genotype (T2308) and three Coffea canephora genotypes (FRT32, FRT05 and FRT64) were used.

The Coffea arabica (T2308, 04-2003) tissues (roots, branches, young leaves, flowers and cherries at different stages of development) and young leaves of Coffea canephora FRT32 were harvested from trees grown in the greenhouse (25° C. and 70% relative humidity) and kept at −80° C. before use. Coffea canephora (FRT32, 2001) cherries, branches, roots and flowers were harvested from trees cultivated in Indonesia. The development stages of the cherries are defined as follows: small green fruit (SG), Large green fruit (LG), yellow fruit (Y) and red fruit (R). The samples were frozen immediately in liquid nitrogen, for shipment prior to use.

Coffea canephora (robusta) FRT05 and FRT64 cherries were harvested from field grown trees in Ecuador, then frozen immediately at −20° C. for shipment prior to use. Subsequently, all samples were stored at −80° C. until use.

RNA extraction. Total RNA was extracted and treated as described previously (Lepelley et al., 2007, Plant Science 172, 978-996), using powders homogenized in a SPEX CertiPrep 6800 Freezer Mill with liquid nitrogen that were stored at −80° C. from the various tissues of Coffea arabica T2308, Coffea canephora FRT32, FRT05 and FRT64 described above in the “Plant Material” section. In the case of the coffee cherries from the different stages, these were first separated into pericarp and grain tissues and then the RNA was extracted from each as described above.

cDNA synthesis. The method used to make the cDNA was identical to the protocol described in the Superscript III Reverse Transcriptase kit (Invitrogen) except either 100 ng of poly dT (18) (Sigma) was used for T2308 and FRT32 or 75 ng of random primers (Invitrogen) was used for FRT05 and FRT64. The cDNA samples generated were then diluted one hundred fold in sterilised water and stored at −20° C. for later use in Q-PCR. Briefly, for the preparation of specific cDNA, 1 μg of total RNA and oligo dT (above) were dissolved in DEPC-treated water (12 μl final volume). This mixture was subsequently incubated at 70° C. for 10 min and then rapidly cooled down on ice. Next, 4 μl of 5× first strand buffer (Invitrogen), 2 μl of DTT 0.1M (Invitrogen) and 1 μl of dNTP mix (10 mM each, Invitrogen), were added. These reaction mixes were preincubated at 42° C. for 2 min before adding 1 μl of SuperScript III Rnase H-Reverse transcriptase (200 U/□μA, Invitrogen). Subsequently, the tubes were incubated at 25° C. for 10 min then at 42° C. for 50 min, followed by enzyme inactivation by heating at 70° C. for 10 min. Finally, 1 U of RNase H (Invitrogen) was added to the reaction mixes, followed by an incubation at 37° C. for 30 min. The cDNA samples generated were then diluted one hundred fold in sterilised water and stored at −20° C. for later use in QPCR.

cDNA libraries. A set of Coffea canephora (robusta) cDNA libraries has been generated as part of collaboration between Nestlé and Cornell University. Over 62,000 cDNA clones from the various libraries were isolated and subjected to 5′ end sequencing to generate ESTs (Expressed Sequence Tags) representing C. canephora genes being expressed in young leaves, and in developing pericarp tissues (all stages mixed), and developing grain (several distinct stages). After quality evaluation, 46,914 high quality ESTs remained and these sequences were then assembled into a unique set of ‘in silico’ coffee gene sequences (‘unigene’ set, ie. the set of unique, non-overlapping coffee cDNA DNA sequences). Details concerning the construction of these libraries, and the bioinformatic analysis of the EST data generated, have been published previously (Lin et al., 2005, Theor. Appl. Genet. 112, 114-130).

DNA sequence analysis. Plasmid DNA were purified from the host using Qiagen kits according to the instructions given by the manufacturer. Prepared plasmid DNA and PCR products were sequenced by GATC Biotech AG (Konstanz, Germany) using the dideoxy termination method. Computer analyses were performed using Laser Gene software package (DNASTAR). Sequence homologies were verified against GenBank databases using the BLAST programs located at the Sol site (http://www.sgn.cornell.edu) and at the NCBI BLAST server (http://blast.ncbi.nlm.nih.gov/Blast.cgi)

Real time qRT-PCR. The cDNA used for these experiments was prepared as described above. Quantitative PCR using TaqMan probes was carried out as described earlier. (Simkin et al., 2006, Journal of Plant Physiology, 163, 691-708) on the Q-PCR machine Applied 7500; except the cDNA dilutions and the Taqman primers/probes were different. A 100 fold dilution of the cDNA was used for all the samples, corresponding to approximately 0.25 ng of original RNA.

The Q-PCR primers and TaqMan probes used were designed with the PRIMER EXPRESS software (Applied Biosybranches) and are listed in Table 1. Numbers in parentheses to the right of each sequence are SEQ ID NOs (e.g., “SID 32”).

TABLE 1

| | | | Efficiency | | |
|---|---|---|---|---|---|
| Gene | Primers and | Primers and Probes Sequences | on | Efficiency on | |
| Names | Probes Names | (5′ → 3′) | plasmids | genomic DNA | |
| rpl39 | rpl39-F1 | GAACAGGCCCATCCCTTATTG (SID 32) | 85% | T2308 | 103% |
| | rpl39-R1 | CGGCGCTTGGCATTGTA (SID 33) | | FRT32 | 99% |
| | rpl39-MGB1 | ATGCGCACTGACAACA (SID 34) | | FRT05 | 97% |
| | | | | FRT64 | 100% |
| UGPP | U348695-F1 | GCAAAACCTGGAACCAAGTTAGAA (SID 35) | 93% | T2308 | 106% |
| | U348695-R1 | GCCATTTATAACCTTGTCAGCAATT (SID 36) | | FRT32 | 105% |
| | U348695-MGB1 | TTCCCGACAGAGCTG (SID 37) | | FRT05 | 96% |
| | | | | FRT64 | 102% |
| GMPP | U352112-F1 | GTGTGGTTGAGGCAGGTGTTAG (SID 38) | 102% | T2308 | 95% |
| | U352112-R1 | GATGCGAACTCCACGCATT (SID 39) | | FRT32 | 92% |
| | U352112-MGB1 | CTCTCACGCTGCACGG (SID 40) | | FRT05 | 94% |
| | | | | FRT64 | 96% |
| PMM | U351352-F1 | GGTGAAGAAAAGCTCAAGGAGTTTA (SID 41) | 97% | T2308 | — |
| | U351352 R1 | TGGGATGTCCAAGTCAGCAA (SID 42) | | FRT32 | — |
| | U351352-MGB1 | AACTTCACGCTCCATTAT (SID 43) | | FRT05 | — |
| | | | | FRT64 | — |
| UGE1 | U347952-F1 | TGTTCAATTCCTAGCATTGTGTTAATACT (SID 44) | 93% | T2308 | 103% |
| | U347952-R1 | CAGGAGGACCATCACGTTTGAGT (SID 45) | | FRT32 | 91% |
| | U347952-MGB1 | TTGGAAGCAAAATC SID 46) | | FRT05 | 106% |
| | | | | FRT64 | 93% |
| UGE5 | U352564-F1 | TGTATGGTTCAGACTCTGAATGGAA (SID 47) | 95% | T2308 | 103% |
| | U352564-R1 | TGTGCACCAACCGGATTG (SID 48) | | FRT32 | 91% |
| | U352564-MGB1 | ATCATATTGCTGCGGTACT (SID 49) | | FRT05 | 106% |
| | | | | FRT64 | 93% |

Quantification was carried out using the method of relative quantification, using the constitutively expressed ribosomal protein rp139 as the reference. In order to use the method of relative quantification, it was necessary to show that the amplification efficiency for the gene sequences was roughly equivalent to the amplification efficiency of the reference sequence (rp139 cDNA sequence) using the specifically defined primer and probe sets. To determine this relative equivalence, plasmid DNA from the coffee databank containing the appropriate cDNA sequences were diluted 1/1000, 1/10,000, 1/100,000, and 1/1,000,000 fold, and using the Q-PCR conditions described above, the slope of the curve Ct=f(Log quantity of DNA) was calculated for each plasmid/primer/TaqMan probe set (Table 1). The plasmids used for determining the efficiencies were: pcccs30w21o13 for rp139, pcccs46w918 for UGPP, pccc122i19 for GMPP, pcccs46w3a14 for PMM, pcccs30w33c4 for UGE1 and pccc117j24 for UGE5.

In order to finalize the validation, all the primer/TaqMan probe sets were tested on the genomic DNA corresponding to the different genotypes used in Q-PCR expression. For this, genomic DNA was extracted from young leaves from genotypes T2308, FRT32, FRT05 and FRT64 (listed above) using DNeasy Plant Maxi Kit (QIAGEN) (Table 1).

Plasmid/primer/TaqMan probe sets giving curves with slopes close to 3.32, which represents an efficiency of 100%, were considered acceptable. The plasmid/primer/TaqMan probe sets used are presented in Table 1 and all gave acceptable values for Ct=f(Log quantity of DNA). All MGB Probes were labelled at the 5′ end with the fluorescent reporter dye 6-carboxyfluorescein (FAM) and at the 3′ with quencher dye 6-carboxy-tetramethyl-rhodamine (TAMRA), except RPL39 probe which was labelled at the 5′ end with the fluorescent reporter dye VIC and at the 3′ end with quencher TAMRA.

Over-expression, purification and activity assay of UGPP and UGE5. The Gateway technology (Invitrogen) composed of the two vectors: the entry vector pENTR/D-TOPO and the expression vector pDEST17, was used to over-produce the UGPP and UGE5 coffee proteins. The strategy consisted of transferring the ORF of UGPP (contained in the pcccs46w918) or UGE5 (contained in the pccc117j24) into the first vector (pENTR/D-TOPO) in frame with an HisTag sequence located in N-terminal. Two specific primers were designed for each construct (based on pcccs46w918 and pccc117j24 insert sequences) to accomplish this. The sense primers (CcUGPP-Forward Primer and CcUGE5-Forward Primer), Table 2, include the specific sequence for the first few codons of the ORF (beginning with the start codon ATG) and the CACC adaptor necessary to direct cloning in pENTR/D-TOPO (5′ to the ATG codon). The reverse primers (CcUGPP-Reverse Primer and CcUGE5-Reverse Primer), Table 2, contain the stop codon of the ORF and several bases from the 3′ UTR. Numbers in parentheses to the right of each sequence are SEQ ID NOs (e.g., “SID 50”).

TABLE 2

| Genes | | Primers and Probes Sequences | Primers |
|---|---|---|---|
| Names | Primers Names | (5′ → 3′) | Lengths |
| UGPP | CcUGPP-Forward Primer | CACCATGGCAACTGCCGCGACT (SID 50) | 22 bp |
| | CcUGPP-Reverse Primer | TTAAATATCCTCAGGGCCATT (SID 51) | 21 bp |
| UGE5 | CcUGE2-Forward Primer | CACCATGCCGGAGAAGATGAAT (SID 52) | 22 bp |
| | CcUGE2-Reverse Primer | TCAATCGGTAGAATCAGGTGAT (SID 53) | 22 bp |

Then, a PCR reaction was performed with the specific primers described above and Pfu Turbo DNA polymerase (Statagene), which does not generate an adenine at the 5′ end of the product and allows the direct cloning of CcUGPP and CcUGE5 PCR products into pENTR/D-TOPO. The PCR amplifications were carried out in a final 50 μl volume, as follows: 1 μl of pcccs46w918 or pccc117j24 plasmid (1/10 diluted), 5 μL 10×PCR buffer (cloned Pfu Reaction Buffer), 400 nM of both specific primers, 200 μM each dNTP, and 1.25 U of Pfu Turbo DNA polymerase (Stratagene). The PCR cycling conditions were as follows: 94° C. for 2 min; then 35 cycles of 94° C. for 1 min, annealing temperature 55° C. for 1 min 30, and 72° C. for 1 min 30. An additional final step of elongation was done at 72° C. for 7 min. The inserts were then cloned into the pENTR/D-TOPO vector following the instructions given by the manufacturer (Invitrogen). This experiment put the CcUGPP and the CcUGE5 ORF into pENTR/D-TOPO vectors (Kanamycin resistance) to form the plasmids pGT38 and pGT25, respectively. The cloning of the inserts was verified by sequencing with M13-RP and M13-FP universal primers that confirmed the correct cloning with no error during the PCR.

Next, pGT38 and pGT25 were recombined with pDEST17 (ampicillin resistance) according to the protocol GATEWAY suggested by the manufacturer (Invitrogen) to produce pGT3 and pGT2, respectively, in which the ORF is in frame with the N-terminal His-Tag in pDEST17. The products of the recombination were transformed into competent cells Top10 (Invitrogen). The ampicillin resistant positive clones were verified to contain the CcUGPP or the CcUGE5 inserts by PCR screening with the specific primers CcUGPP-Forward Primer/CcUGPP-Reverse Primer or CcUGE5-Forward Primer/CcUGE5-Reverse Primer described in Table 2. After purification pGT3 and pGT2 were then transformed in competent cells BL21-AI™ OneShot® Chemically Competent E. coli (Invitrogen) (for protein expression) according to the protocol suggested by the supplier (Invitrogen). The cloning was then verified by sequencing with the T7 universal primer which showed that CcUGPP and CcUGE5 were in frame with the N-terminal His tag.

For protein expression, 2 mL of cultures of B121AI cells transformed with pGT3 or pGT2 (40% glycerol) were grown around 3 hours at 37° C. and 200 rpm in 100 ml of LB medium containing 100 μg/ml of ampicillin to an OD600 nm=0.6. 1 mL of the culture was then kept to be for subsequent protein extraction and visualized on SDS Page gel. The expression of the cloned protein was then induced with 0.2% of L-arabinose and the culture was incubated for a further 2 h at 27° C. The 1 mL of the induced culture was kept for extraction and SDS Page gel.

The cells were pelleted at 5500 g for 30 min at 4° C., then the bacterial pellet harvested was resuspended in 5 mL of BugBuster® Protein Extraction Reagent (Novagen) to which 5 μL of Benzonase® Nuclease (Novagen) and protease inhibiteurs Complete Mini EDTA-free (Roche) were added. After a 30 min incubation at room temperature at 70 rpm, the lysed cells were centrifuged at 10,000 g for 30 min at 4° C. in order to obtain the soluble proteic extract (supernatant) and the insoluble protein fraction (pellet).

Fifteen (15) μL of the collected induced/non induced extracts and the soluble/insoluble protein extracts were then vizualized, with the Prestained SDS-PAGE Low Range molecular weight standards (BIO-RAD) on a 8-16% Acrylamide Express PAGE Gels (GenScript Corp.) using the denaturing buffer 5× Sample Buffer (GenScript Corp.). The migration buffer used was the Tris-HEPES-SDS Running Buffer (GenScript Corp.), at 100 V. The gel was then colored 20 min at 70 rpm with the coloration solution (0.25% w/v Coomassie blue, 10% acetic acid and, 20% ethanol), then washed twice for 20 min at 70 rpm with the strong decoloration solution (40% ethanol, 7% acetic acid), and then washed one time overnight at 70 rpm using a low decoloration solution (10% ethanol, 10% acetic acid, 5% glycerol).

### Example 2

### Isolation and Characterization of cDNA Encoding Coffea canephora PMM, GMPP, UGPP and UGE

This example describes the isolation and characterization of cDNA sequences encoding proteins directly involved in the synthesis of key precursors for galactomannan synthesis, UDP-galactose and GDP-mannose. The selected enzymes were PMM (phosphomannomutase), GMPP (GDP-mannose pyrophosphorylase), UGPP (UDP-glucose pyrophosphorylase) and UGE (UDP-glucose 4-epimerase). Various BLAST programs (see Example 1) were used to search for unigene sequences with the highest similarity to public database protein sequences encoding biochemically characterized PMM, GMPP, UGPP and UGPP proteins. Except for UGE1, the longest cDNA of each “best unigene hit” was then selected for full sequencing. Results are summarized in Table 3 and Table 4.

Table 3 references the UGPP, GMPP, PMM and UGE protein sequences from other organisms than coffee that have been used to identify the coffee Unigenes by Blast at http://www.sgn.cornell.edu. Tblastn identities result from blast performed using a full protein sequence as query against the database containing the nucleotides sequences of all coffea canephora Unigenes translated to proteins. Blastn identities result from blast performed using a full coding sequence (CDS) as query against the database containing the nucleotides sequences of all Coffea canephora Unigenes.

Table 4 sets out a list of the Coffea canephora Unigenes identified at http://www.sgn.cornell.edu as potentially encoding an UGPP, a GMPP, a PMM and two UGE coffee proteins. The names of the clones that were entirely characterized and sequenced to confirm the “in silico” sequences of the identified Unigenes are indicated, as well as the number of ESTs found in each Unigene. The SGN ID correspond to the SGN numbers attributed to the Unigenes sequences from Coffea canephora Built #2 accessible on the SGN Website.

TABLE 3

| | | | CDS | | Protein | | |
|---|---|---|---|---|---|---|---|
| | | | accession | Blastn | accession | tBlastn | SGN Unigene |
| Function | Organism | Name | Number | identities | Number | identities | Number |
| UGPP | Cucumis melo | CmUGPP | DQ445483 | 82% | ABD98820 | 86% | SGN-U348695 |
| | Oryza sativa | OsUGPP | DQ395328 | 81% | ABD57308 | 87% | |
| | Solanum tuberosum | StUGPP | Z18924 | 81% | CAA79357 | 87% | |
| | Arabidopsis thaliana | AtUGPP | AF361605 | 83% | AAK32773 | 87% | |
| GMPP | Solanum tuberosum | StGMPP | AF022716 | 84% | AAD01737 | 91% | SGN-U352112 |
| | Solanum lycopersicum | SlGMPP | AY605668 | 83% | AAT37498 | 90% | |
| | Arabidopsis thaliana | AtGMPP | AF076484 | 80% | AAC78474 | 92% | |
| | Medicago sativa | MsGMPP | AY639647 | 82% | AAT58365 | 92% | |
| | Vitis vinifera | VvGMPP | CU459234 | 83% | CAO69137 | 93% | |
| PMM | Arabidopsis thaliana | AtPMM | DQ442991 | 79% | ABD97870 | 81% | SGN-U351352 |
| | Oryza sativa | OsPMM | DQ442992 | 82% | ABD97871 | 89% | |
| | Solanum lycopersicum | SlPMM | DQ442993 | 83% | ABD97872 | 87% | |
| | Glycine max | GmPMM | DQ442994 | 83% | ABD97873 | 91% | |
| | Nicotiana tabacum | NtPMM | DQ442995 | 83% | ABD97874 | 90% | |
| | Triticum aestivum | TaPMM | DQ442996 | 80% | ABD97875 | 88% | |
| UGE | Arabiopsis thaliana | AtUGE1 | NM_101148 | 80% | NP_172738 | 82% | SGN-U347952 |
| | | | | | | 64% | SGN-U352564 |
| | Arabidopsis thaliana | AtUGE3 | NM_104996 | 78% | NP_564811 | 79% | SGN-U347952 |
| | | | | | | 66% | SGN-U352564 |
| | Solanum tuberosum | StUGE51 | AY221085 | 82% | AAP97493 | 81% | SGN-U347952 |
| | | | | | | 65% | SGN-U352564 |
| | Populus trilocarpa | PtUGE | EF147280 | 82% | ABK95303 | 87% | SGN-U352564 |
| | | | | | | 65% | SGN-U347952 |
| | Solanum tuberosum | StUGE45 | AY197749 | 83% | AAP42567 | 86% | SGN-U352564 |
| | | | | | | 68% | SGN-U347952 |
| | Vitis vinifera | VvUGE | AM459205 | 85% | CAN63477 | 86% | SGN-U352564 |
| | | | | | | 65% | SGN-U347952 |
| | Arabidopsis thaliana | AtUGE2 | NM_118524 | 84% | NP_194123 | 80% | SGN-U352564 |
| | | | | | | 62% | SGN-U347952 |
| | Arabidopsis thaliana | AtUGE4 | NM_105119 | 86% | NP_176625 | 78% | SGN-U352564 |
| | | | | | | 62% | SGN-U347952 |

TABLE 4

| | Gene | | Clone | |
|---|---|---|---|---|
| Annotation | name | SGN ID | name | Number of ESTs |
| UDP-glucose | UGPP | SGN- | cccs46w918 | 9 (grain 46 w) |
| pyrophos- | | U348695 | | + 2 (pericarp) |
| phorylase | | | | + 8 (leaves) |
| | | | | + 3 (whole cherries) |
| GDP-mannose | GMPP | SGN- | ccc122i19 | 7 (grain 46 w) |
| pyrophos- | | U352112 | | + 8 (leaves) |
| phorylase | | | | |
| Phospho- | PMM | SGN- | cccs46w3a14 | 1 (grain 30 w) |
| mannomutase | | U351352 | | + 5 (grain 46 w) |
| | | | | + 2 (pericarp) |
| | | | | + 1 (leaves) |
| UDP-Glucose | UGE1 | SGN- | cccs30w33c4 | 2 (grain 30 w) |
| 4-epimerase | | U347952 | | + 2 (whole cherries) |
| | UGE5 | SGN- | cccl17j24 | + 3 (leaves) |
| | | U352564 | | |

A. UDP-Glucose Pyrophosphorylase (CcUGPP)

To find a coffee cDNA encoding the enzyme UDP-Glucose pyrophorylase (UGPP), two protein sequences encoding biochemically characterized UGPP proteins, the Oryza sativa UDP-Glucose pyrophorylase (Chen et al., 2007, Plant Cell 19, 847-861; accession number ABD57308) and the Cucumis melo UDP-Glucose pyrophorylase (Dai et al., 2006, Plant Physiol 142, 294-304; accession number ABD98820) were used to search the Nestlé/Cornell ‘unigene’ Built2 with the tblastn algorithm. This search uncovered one unigene (SGN-U348695) exhibiting homology to the O. sativa and C. melo UGPP protein sequences (87% and 86% identity, respectively, with e-value=0

A cDNA representing the 5′ end of the unigene SGN-U348695 (pcccs46w918), and potentially encoding the full ORF of this protein, was then isolated and sequenced. This Unigene comprises nine ESTs isolated from the grain at 46 weeks after flowering, two from the pericarp, eight from the leaves and three from the cherries of different developmental stages (Table 4).

The insert of pcccs46w918 was 1750 bp long, and encodes an ORF of 1434 bp. The deduced protein sequence comprises 477 amino acids, and has a predicted molecular weight of 52.49 kDa. An optimized alignment (ClustalW) of the protein sequence of pcccs46w918 (CcUGPP) with UGPP protein sequences from A. thaliana, C. melo, O. sativa and an orthologous sequence from S. tuberosum demonstrates that the protein encoded by pcccs46w918 shares, respectively, 81.7%, 86.8%, 87% and 87.8% identity with these protein sequences (FIG. 1 and Table 5).

TABLE 5

| | Percent Identity | | | | | | |
|---|---|---|---|---|---|---|---|
| | 1 | 2 | 3 | 4 | 5 | | |
| 1 | | 87.8 | 87.0 | 86.8 | 81.7 | 1 | CcUGPP |
| 2 | | | 86.1 | 85.5 | 82.9 | 2 | StUGPP CAA79357 |
| 3 | | | | 85.1 | 83.3 | 3 | OsUGPP ABD57308 |
| 4 | | | | | 82.9 | 4 | CmUGPP ABD98820 |
| 5 | | | | | | 5 | AtUGPP AAK32773 |
| | 1 | 2 | 3 | 4 | 5 | | |

The alignment data indicate that pcccs46w918 encodes a full length cDNA for a C. canephora UDP-Glucose pyrophorylase (CcUGPP). An optimized alignment (Jotun Hein Method) of the DNA sequence encoding the full CDS sequence contained in pcccs46w918 with DNA sequences encoding the full CDS sequences of UGPP from A. thaliana, C. melo, O. sativa and S. tuberosum demonstrated that the ORF DNA sequence of the coffee CcUGPP shares 75.1%, 78.8%, 77.1% and 80.6% identity with these CDS DNA sequences, respectively.

B. GDP-Mannose Pyrophosphorylase (CcGMPP)

To find a cDNA encoding a coffee GMPP, the biochemically characterized S. tuberosum GMPP protein sequence (accession number AAD01737; (Keller et al., 1999, Plant J. 19(2), 131-141) served as the query sequence for a tBLASTn search against the Nestlé/Cornell ‘unigene’ Built2 with tblastn algorithm (Table 3). The best match obtained was unigene SGN-U352112 (e value=1e-163, Score=567 bits (1462), Identities=283/310 (91%)). This Unigene comprises seven ESTs isolated from the grain at 46 weeks after flowering and eight from the leaves (Table 4).

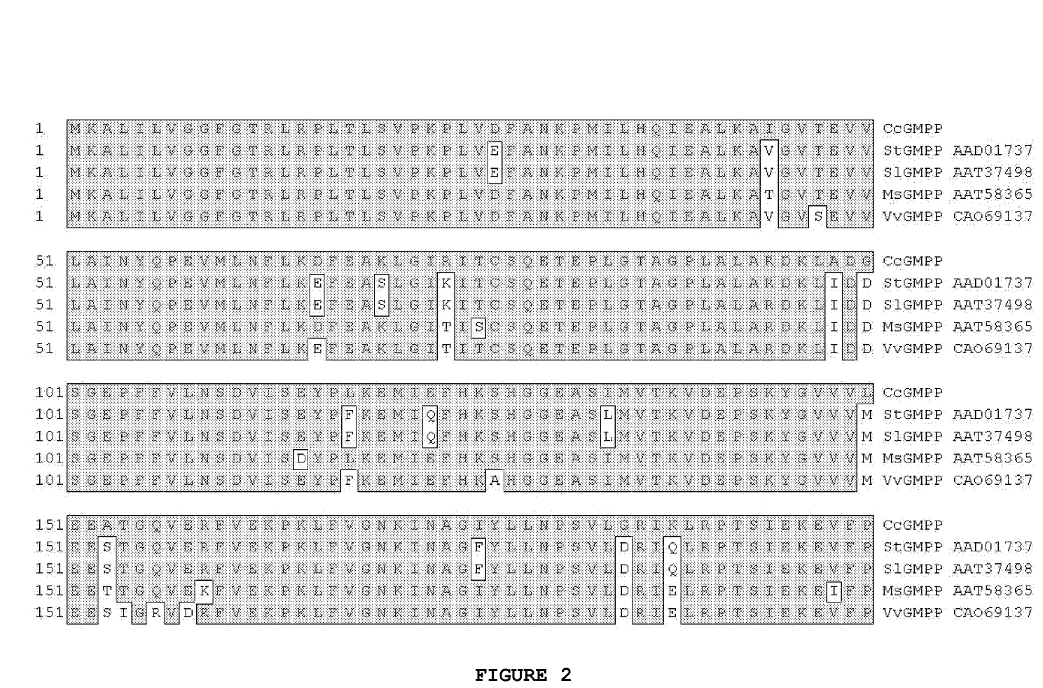

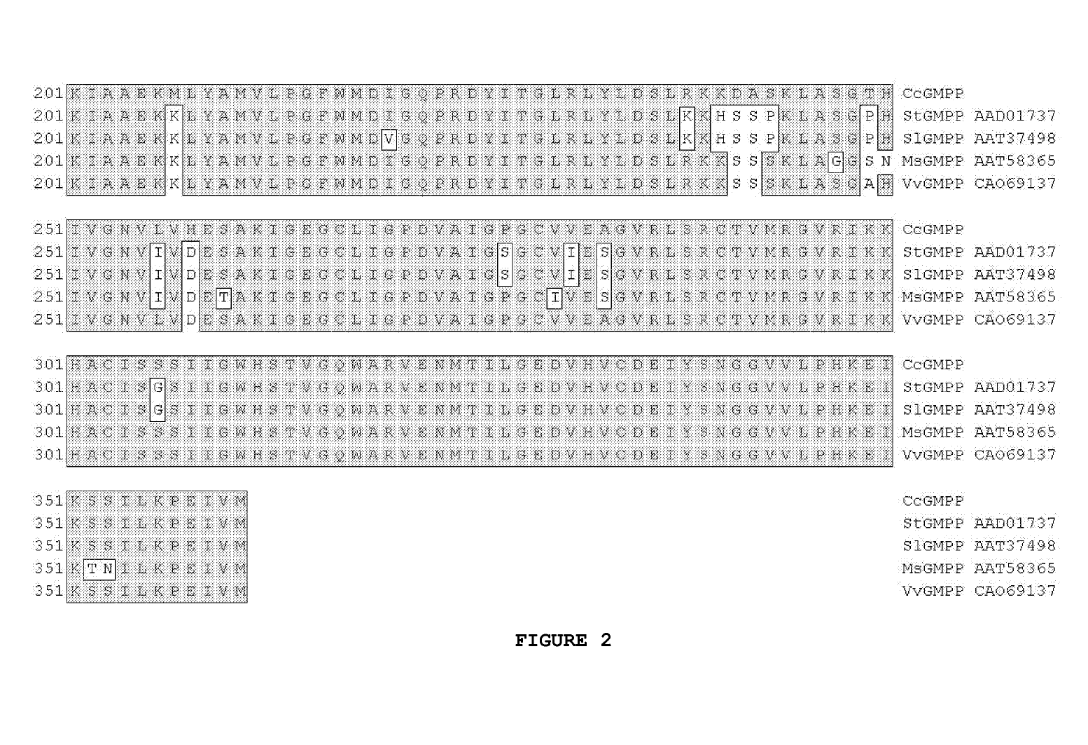

A cDNA representing the 5′ end of unigene SGN-U352112 (pccc122i19), and thus encoding the longest coffee cDNA in the Nestlé/Cornell database related to the potato GMPP, was isolated and sequenced. The insert of pccc122i19 was found to be 1576 bp long and comprised a full CDS sequence of 1086 bp encoding a protein of 361 amino acids (estimated molecular weight of 39.43 kDa). Alignment of the complete of the coffee protein sequence CcGMPP encoded by pccc122i19 with protein sequence of S. tuberosum, S. lycopersicum, M. sativa and Vitis vinifera (accession numbers AAD01737, AAT37498, AAT58365 and CA069137 respectively) confirms the initial annotation of this coffee sequence using ClustalW, i.e., the CDS of pccc122i19 encodes a coffee GMPP protein (FIG. 2 and Table 6).

TABLE 6

| | Percent Identity | | | | | | |
|---|---|---|---|---|---|---|---|
| | 1 | 2 | 3 | 4 | 5 | | |
| 1 | | 92.2 | 92.0 | 93.1 | 94.5 | 1 | CcGMPP |
| 2 | | | 99.7 | 92.2 | 94.2 | 2 | StGMPP AAD01737 |
| 3 | | | | 92.0 | 93.9 | 3 | SlGMPP AAT37498 |
| 4 | | | | | 93.9 | 4 | MsGMPP AAT58365 |
| 5 | | | | | | 5 | VvGMPP CAO69137 |
| | 1 | 2 | 3 | 4 | 5 | | |

At the protein level, this coffee GMPP sequence exhibits 92.2%, 92%, 93.1% and 94.5% identity with S. tuberosum, S. lycopersicum, M. sativa and Vitis vinifera GMPP protein sequences. At the nucleic level, still using ClustalW method, the complete CDS of the coffee sequence exhibits 83.5%, 82.7%, 81.4% and 84% identity with S. tuberosum, S. lycopersicum, M. sativa and Vitis vinifera complete CDS sequences respectively. It should be noted that the identity data at the DNA level is only for the CDS sequence, thus it probably over-estimates the similarity of the complete cDNA sequences due to the lower levels of identity generally associated the 5′ and 3′ UTR sequences of cDNA.

C. Phosphomannomutase (CcPMM)

To find a cDNA encoding a coffee Phosphomannomutase, the biochemically characterized A. thaliana phosphomannomutase protein sequence (accession number ABD97870; Qian et al., 2007, Plant J. 49(3), 399-413) served as the query sequence for a tblastn search against the Nestlé/Cornell ‘unigene’ (Table 3). The best hit obtained was unigene SGN-U351352 (e value=1e-111, Score=395 bits (1014), Identities=190/233 (81%)). This Unigene comprises six ESTs isolated from the grain (one at 30 weeks after flowering and five at 46 weeks after flowering), two from the pericarp and one from the leaves (Table 4).

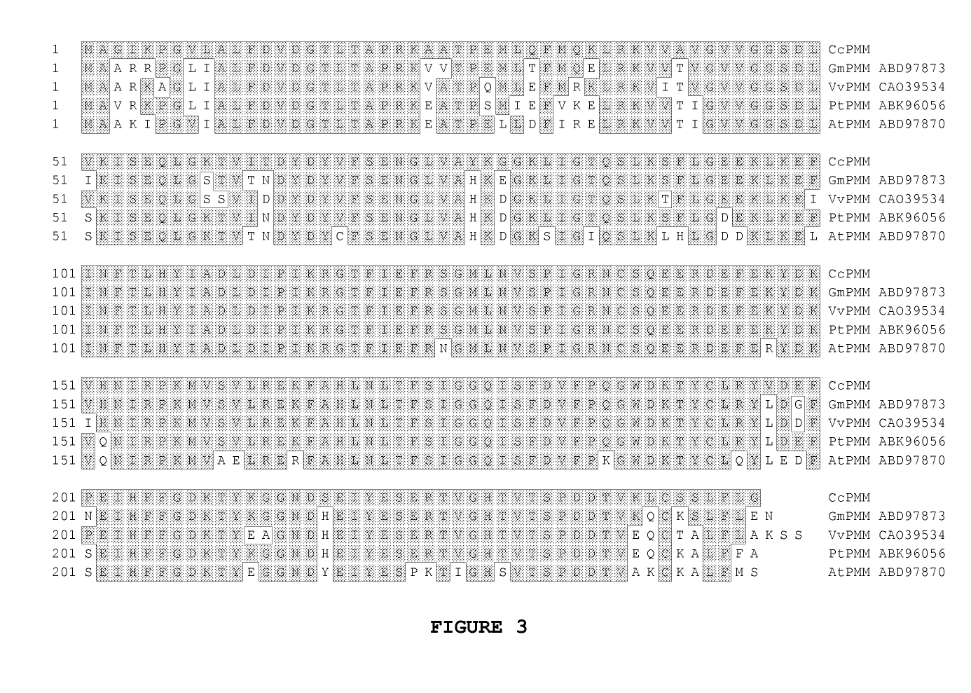

A cDNA representing the 5′ end of unigene SGN-U351352 (pcccs46w3a14), and thus encoding the longest coffee cDNA in the Nestlé/Cornell database related to the Arabidopsis PMM, was isolated and sequenced. The insert of pcccs46w3a14 was found to be 1218 bp long and comprised a full CDS sequence of 741 pb encoding a protein of 246 amino acids (estimated molecular weight of 27.59 kDa). Alignment of the complete coffee protein sequence of CcPMM encoded by pcccs46w3a14 with protein sequence of G. max, V. vinifera P. trichocarpa and A. thaliana (accession numbers ABD97873, CA039534, ABK96056 and ABD97870 respectively) confirms the initial annotation of this coffee sequence using ClustalW, i.e., the CDS of pcccs46w3a14 encodes a coffee PMM protein (FIG. 3). At the protein level, this coffee PMM sequence exhibits 90.7%, 88.2%, 88.6% and 80.1% identity with G. max, V. vinifera P. trichocarpa and A. thaliana PMM protein sequences (Table 7).

TABLE 7

| | Percent Identity | | | | | | |
|---|---|---|---|---|---|---|---|
| | 1 | 2 | 3 | 4 | 5 | | |
| 1 | | 90.7 | 88.2 | 88.6 | 80.1 | 1 | CcPMM |
| 2 | | | 89.9 | 91.1 | 82.5 | 2 | GmPMM ABD97873 |
| 3 | | | | 89.8 | 80.9 | 3 | VvPMM CAO39534 |
| 4 | | | | | 85.0 | 4 | PtPMM ABK96056 |
| 5 | | | | | | 5 | AtPMM ABD97870 |
| | 1 | 2 | 3 | 4 | 5 | | |

At the nucleic level, still using ClustalW method, the complete CDS of the coffee sequence exhibits 80.4%, 79.6%, 78.9% and 71.8% identity with G. max, V. vinifera, P. trichocarpa and A. thaliana complete CDS sequences respectively. It should be noted that he identity data at the DNA level is only for the CDS sequence, thus it probably over-estimates the similarity of the complete cDNA sequences due to the lower levels of identity generally associated the 5′ and 3′ UTR sequences of cDNA.

D. Two cDNA Clones Encoding UDP-Glucose 4-Epimerases (CcUGE1, CcUGE5)

In order to identify coffee cDNA encoding UDP-Glucose 4-epimerases in Coffea canephora EST databank, the biochemically characterized A. thaliana UGE5 protein sequence (accession number NP_{—}194123; Rösti et al., 2007, Plant Cell 19 (5), 1565-1579) served as the query sequence for a tblastn search against the Nestlé/Cornell ‘unigene’ Built2 (Table 3). In Arabidopsis, AtUGE5 has been shown to influence growth and cell wall carbohydrate biosynthesis throughout the plant (Rösti et al., 2007, supra). The two best hits obtained were unigenes SGN-U352564 (e value=1e-109, Score=390 bits (1003), Identities=209/231 (80%)) and SGN-347952 (e value=1e-127, Score=448 bits (1153), Identities=266/339 (62%)). Unigene SGN-U352564 comprises three ESTs isolated from the leaves and SGN-347952 two from the grain at 30 weeks after flowering and two from whole cherries (Table 4).

A second characterized A. thaliana UGE protein sequence, UGE1 (accession number NP_{—}172738; Rösti et al., 2007, supra) was used as a query sequence to perform a tBlastn against the C. canephora Nestlé/Cornell Unigenes sequences from the Built2. Three hits were obtained, showing more than 50% identities. Again, the two best hits obtained were unigenes SGN-347952 (e value=1e-172, Score=600 bits (1546), Identities=289/350 (82%)) and SGN-U352564 (e value=5e-85, Score=309 bits (791), Identities=151/234 (64%)).

Coffea canephora cDNA clone encoding CcUGE1. The Unigene SGN-U347952 was found to encode a full “in silico” coffee cDNA in the Nestlé/Cornell database related to the Arabidopsis UGE1 sequence. The longest, full cDNA representing the 5′ end of unigene SGN-U351352 was not available. However, another partial cDNA representing from by 696 to 1424 of this unigene (pcccs30w33c4), was isolated and sequenced. The insert of pcccs30w33c4 was found to be 732 bp long and comprised a partial CDS sequence (546 bp long and missing 509 bp from 5′ end), encoding a partial ORF of 181 amino acids (estimated molecular weight of 20.24 kDa).

Alignment of the “in silico” sequence of Unigene SGN-U347952 with the insert pcccs30w33c4 sequence, performed by Seqman software, showed this cDNA sequence and the unigene sequences are 100% identical over 729 bp. The bioinformatic study of the “in silico” sequence from Unigene SGN-U347952 also showed this Unigene has a complete CDS of 1056 bp encoding a 351 aa long protein (estimated molecular weight of 39 kDa). Considering that the sequence of the insert from pcccs30w33c4 and the sequences from the 5′ end clones of unigene SGN-U347952 (cDNA sequences CC-F01_{—}017_L05 and CC-F01_{—}014_P09), all show 100% identity with Unigene SGN-U347952, it can be said that the “in silico” sequence from Unigene SGN-U347952 is accurate and represents an in silico sequence of a single gene. This Unigene sequence was then named CcUGE1 because of its higher level of identity with A. thaliana UGE1 than with the other A. thaliana UGE.

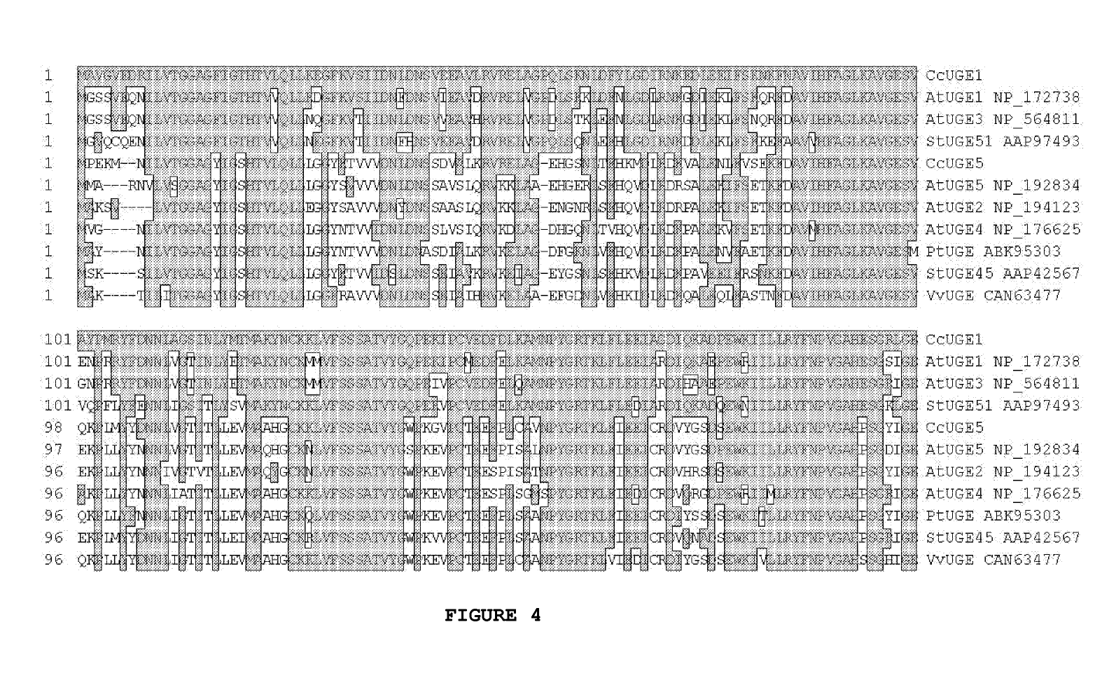

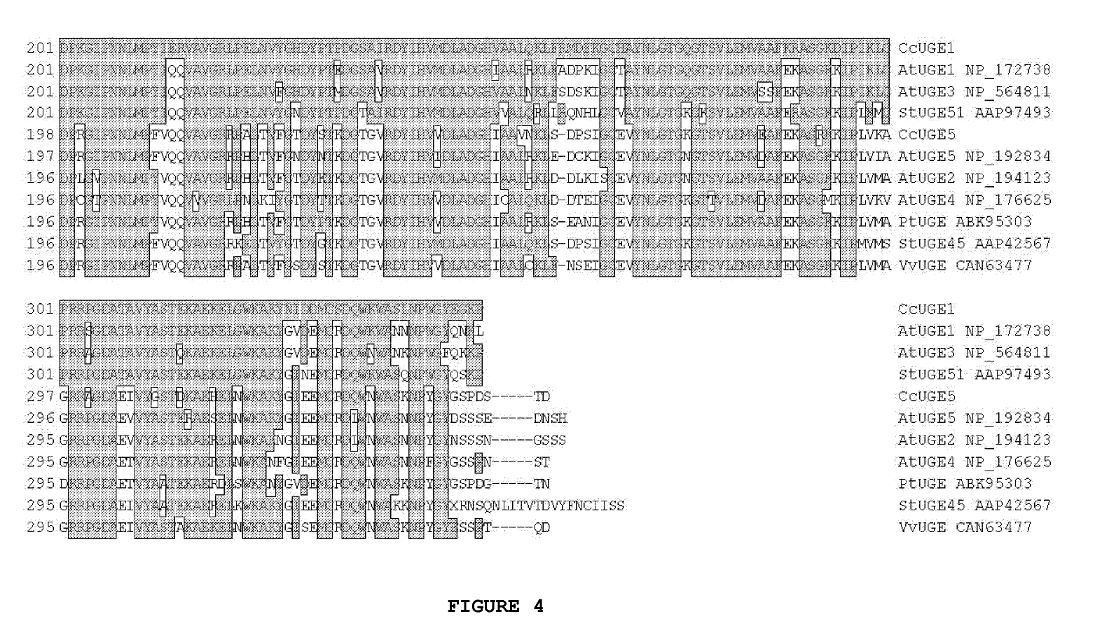

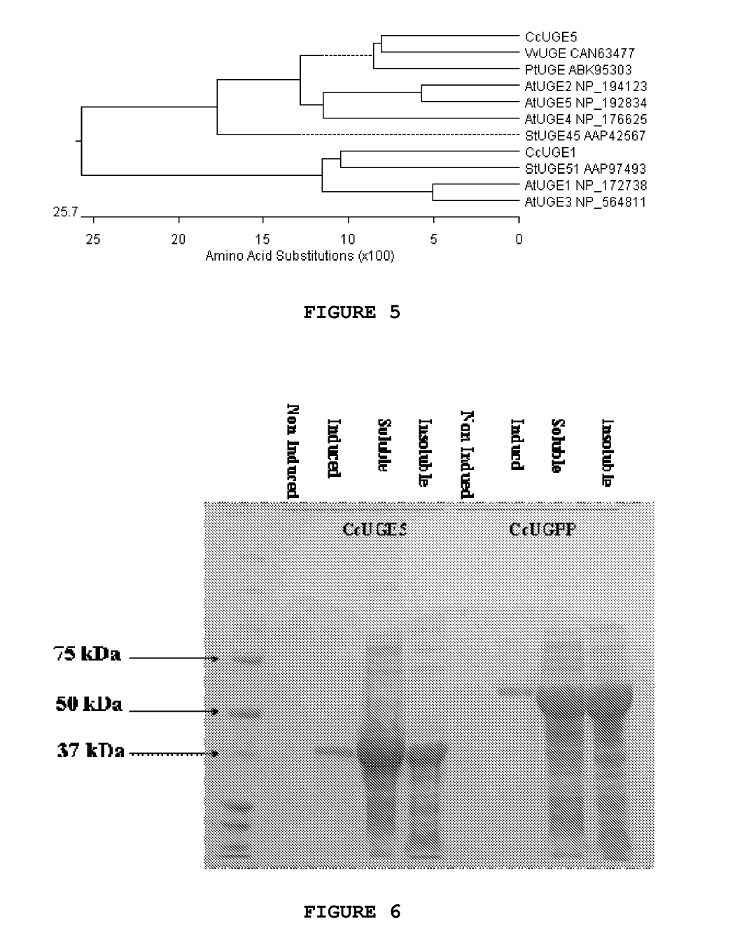

Alignment of the complete coffee protein sequence CcUGE1 encoded by SGN-U347952 with the UGE protein sequences of A. thaliana (UGE1 to UGE5), P. trichocarpa, S. tuberosum and V. vinifera (accession numbers available in FIG. 4) confirms the initial annotation of this coffee sequence using ClustalW, i.e., the CDS of SGN-U347952 encodes a coffee UGE protein (FIG. 4). At the protein level, this coffee CcUGE1 sequence deriving from Unigene SGN-U347952 exhibits the higher levels of identity with A. thaliana UGE1 (82.3%), A. thaliana UGE3 (79.5%) and S. tuberosum UGE51 (81.8%) proteins (FIG. 4, FIG. 5 and Table 8). On the five Arabidopsis UGE protein sequences, AtUGE1 was most closely related to the coffee protein encoded by SGN-U347952, and this latter sequence was thus definitely named CcUGE1 (note: no full length cDNA currently exists for this sequence).

TABLE 8

| | Percent Identity | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | | |
| 1 | | 82.3 | 79.5 | 81.8 | 63.0 | 61.5 | 61.5 | 61.5 | 63.5 | 66.1 | 64.4 | 1 | CcUGE1 |
| 2 | | | 90.6 | 80.3 | 65.8 | 65.2 | 65.2 | 63.5 | 66.1 | 67.2 | 65.0 | 2 | AtUGE1 NP_172738 |
| 3 | | | | 78.6 | 67.0 | 64.7 | 64.4 | 62.7 | 65.8 | 66.4 | 66.4 | 3 | AtUGE3 NP_564811 |
| 4 | | | | | 64.7 | 63.2 | 63.0 | 65.0 | 65.2 | 65.8 | 66.7 | 4 | StUGE51 AAP97493 |
| 5 | | | | | | 81.6 | 79.4 | 78.3 | 84.4 | 83.0 | 85.0 | 5 | CcUGE5 |
| 6 | | | | | | | 87.5 | 79.7 | 81.6 | 77.8 | 80.8 | 6 | AtUGE5 NP_192834 |
| 7 | | | | | | | | 79.7 | 81.3 | 77.8 | 79.1 | 7 | AtUGE2 NP_194123 |
| 8 | | | | | | | | | 80.5 | 76.9 | 77.7 | 8 | AtUGE4 NP_176625 |
| 9 | | | | | | | | | | 81.6 | 84.4 | 9 | PtUGE ABK95303 |
| 10 | | | | | | | | | | | 81.1 | 10 | StUGE45 AAP42567 |
| 11 | | | | | | | | | | | | 11 | VvUGE CAN63477 |
| | 1 | 2 | 3 | 4 | 5 | 6 | 7 | 8 | 9 | 10 | 11 | | |

Coffea canephora cDNA clone encoding CcUGE5. A cDNA representing the 5′ end of unigene SGN-U352564 (pccc117j24) and thus encoding the longest coffee cDNA in the Nestlé/Cornell database related to the Arabidopsis UGE2 sequence, was isolated and sequenced. The insert of pccc117j24 found to be 1434 bp long and comprised a full CDS sequence of 1053 bp encoding a protein of 350 amino acids (estimated molecular weight of 38.42 kDa). Alignment of the complete coffee protein sequence encoded by pccc117j24 with UGE protein sequences of A. thaliana (UGE1 to UGE5), P. trichocarpa, S. tuberosum and V. vinifera (accession numbers available in FIG. 4) confirms the initial annotation of this coffee sequence using ClustalW, i.e., the CDS of pccc117j24 encodes a coffee UGE protein (FIG. 4, FIG. 5 and Table 8). At the protein level, this coffee sequence exhibits the higher levels of identity with VvUGE (85%), PtUGE (84.4%), StUGE45 (83%), AtUGE5 (81.6%), AtUGE2 (79.4%) and AtUGE4 (78.3%) proteins. This coffee protein sequence encoded by pccc117j24 also shares 63% identity with CcUGE1 and was named CcUGE5.

### Example 3

### Over-Expression of Recombinant CcUGPP and CcUGE5

To confirm the annotation of pcccs46w918 (CcUGPP) and pccc117j24 (CcUGE5), these proteins were expressed in recombinant forms in E. coli. As described in Example 1, the Gateway technology cloning system was used to express CcUGPP and CcUGE5. The complete ORFs were first cloned into pENTR/D-TOPO entry vector to form the plasmids pGT38 and pGT25, respectively, then pGT38 and pGT25 were recombined with the pDEST17 destination vector to produce pGT3 and pGT2 plasmids containing the CcUGPP and the CcUGE5 full coding sequences in frame with an N Terminal His-Tag. These two plasmids pGT3 and pGT2 were then transformed into BL21-AI cells and the CcUGPP and CcUGE5 proteins overexpressed using an induction of expression with arabinose. The collected induced/non induced extracts and the soluble/insoluble protein extracts were then visualized on gel. FIG. 6 shows the results of this over-expression experiment and demonstrates that a good induction of the his-tagged proteins UGPP and UGE5 with the approximate size expected (approximately 52.5 kDa and 38.4 kDa, respectively plus 2.6 kDa for the Fusion Tag) occurred after induction of the transformed cells. Strong signals in the soluble and insoluble fraction show that the CcUGPP and CcUGE5 proteins were produced in both fractions, although with a higher production in soluble fraction, especially in the case of CcUGE5.

### Example 4

### Tissue-Specific Expression of PMM, GMPP, UGPP and UGE Genes

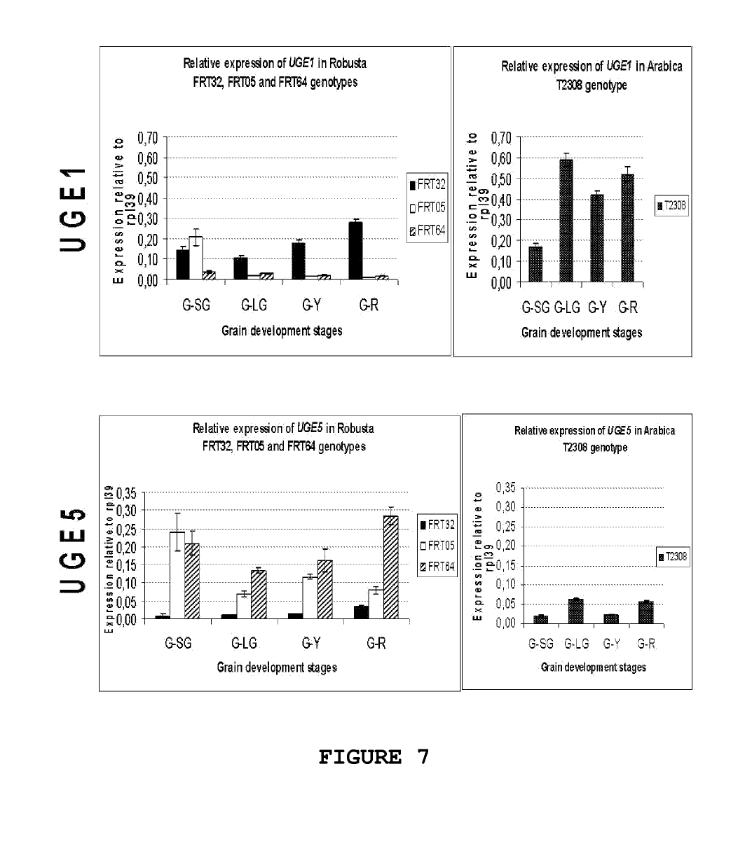

The quantitative expression of transcripts from the PMM, GMPP, UGPP and UGE genes was determined for several tissues of the arabica variety T2308 and of the robusta varieties FRT32, FRT05 and FRT64 using gene specific TaqMan primers/probes (Table 1). The different cDNA for these experiments were prepared by the method described Example 1, with RNA isolated from: (1) the grain and pericarp tissues isolated from 4 different stages of developing arabica T2308 and robusta FRT32, FRT05 and FRT64 coffee cherries; and (2) from roots, branches, leaves and flowers from arabica T2308 and robusta FRT32 as described in the Example 1. The results of these experiments are presented in FIGS. 7, 8 and 9. Quantification was carried out using the method of relative quantification, using the constitutively expressed ribosomal protein rp139 as the reference.

A. Relative Expression of PMM, GMPP, UGPP and UGE During Grain Maturation of Three Robustas (FRT32, FRT05 and FRT64) and Arabica T2308

It is noted that all the primer/probe sets used in this experiment were validated using plasmid based cDNA containing each sequence and were also tested on genomic DNA of each genotype used in these experiments (Table 1). Such experiments ensure that the primers/probes used are efficient in quantitatively measuring the presence of their specific sequences in a simple situation (plasmid DNA) and also recognise the genes in all the plants analysed to approximately the same efficiency, that is, (a) the presence of the gene is confirmed in each genome and (b) this ensures there are no differences in detection due to allelic changes in the sequences being tested. It is further noted that, in the case of the efficiency of the primers/probes on the genomic DNA, the primers/probes set specific to PMM gene did not permit the amplification of genomic DNA. Because this set was able to amplify plasmid DNA with 97% efficiency, it was surmised that the primers and/or probe may have been designed at a junction of an exon and an intron, and thus were not able to amplify genomic DNA. However, given the good results with the cDNA, it was concluded that the primers/probes specific to the PMM gene were acceptable for the Q-RT-PCR experiments described in this example.

B. Comparison of Expression During Grain Development

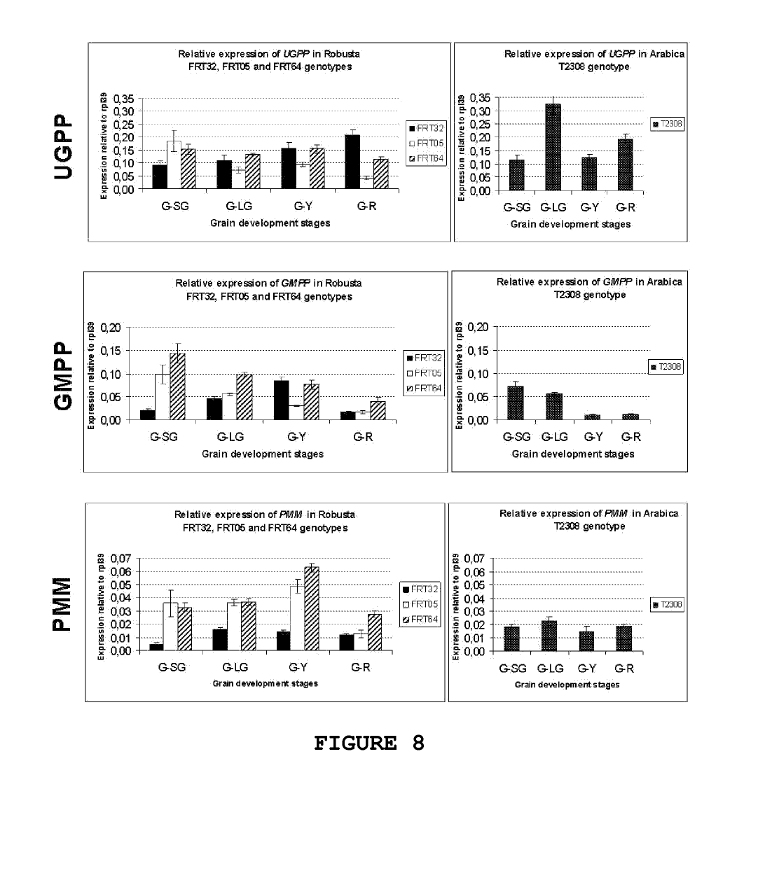

FIG. 8 presents the transcript accumulation profiles from the coffee genes encoding GMPP, UGPP, PMM in the robusta varieties FRT05, FRT64, and FRT32 and in the arabica variety T2308 (CCCA02). The expression profiles and expression levels for the UGPP gene is relatively similar for all four varieties with expression levels having RQ roughly between 0.1 and 0.2. It is noted however that there is a tendency for transcript levels for FRT 32 to rise slightly, and for FRT05 to fall sightly as development progresses. For some genes, there also appears to be a spike in the transcript level at the large green stage of the arabica T2308 grain. The expression profile for PMM is also globally stable during grain development in the different varieties (FIG. 8), although there are some small differences. In general, the expression level of PMM is in the region of RQ 0.1-0.4. However, FRT05 and FRT64 RQ levels are at the higher end of the scale and seem to have a spike at the yellow stage, followed by a drop at the red stage. In arabica the expression level is at the lower end of the scale, but relatively constant throughout the development period. The expression profiles for GMPP are somewhat more complex. Overall, the RQs ranged between approximately 0.01 and 0.144. There appeared to be two distinct patterns of expression, one with quite low expression at early and late stages FRT32, and then the others (FRT05, FRT64 and arabica T2308), where expression was highest in the small green grain and then decreased at each of the following steps. All the varieties had relatively similar levels of GMPP transcripts at the red stage.

The expression data for the two characterized UGE genes is presented in FIG. 7 and set forth in Tables 9 (UGE1) and 10 (UGE5) below.

TABLE 9

| | FRT32 | | FRT05 | | FRT64 | | T2308 | |
|---|---|---|---|---|---|---|---|---|
| | medium | standard | medium | standard | medium | standard | medium | standard |
| Sample | RQ | deviation | RQ | deviation | RQ | deviation | RQ | deviation |
| G-SG | 0.144 | 0.019 | 0.208 | 0.042 | 0.039 | 0.006 | 0.170 | 0.017 |
| G-LG | 0.110 | 0.010 | 0.022 | 0.002 | 0.030 | 0.001 | 0.588 | 0.032 |
| G-Y | 0.176 | 0.017 | 0.016 | 0.001 | 0.023 | 0.002 | 0.418 | 0.024 |
| G-R | 0.279 | 0.017 | 0.010 | 0.001 | 0.018 | 0.004 | 0.521 | 0.037 |

TABLE 10

| | FRT32 | | FRT05 | | FRT64 | | T2308 | |
|---|---|---|---|---|---|---|---|---|
| | medium | standard | medium | standard | medium | standard | medium | standard |
| Sample | RQ | deviation | RQ | deviation | RQ | deviation | RQ | deviation |
| G-SG | 0.010 | 0.004 | 0.240 | 0.053 | 0.208 | 0.033 | 0.018 | 0.003 |
| G-LG | 0.011 | 0.001 | 0.069 | 0.008 | 0.134 | 0.006 | 0.062 | 0.004 |
| G-Y | 0.013 | 0.001 | 0.117 | 0.007 | 0.163 | 0.032 | 0.021 | 0.003 |
| G-R | 0.035 | 0.004 | 0.080 | 0.010 | 0.284 | 0.025 | 0.057 | 0.003 |

UGE1 appears to exhibit two types of expression patterns, with both patterns of expression having a range of expression levels between RQ 0.01 and 0.28 for the robusta and RQ 0.17-0.59 for the single arabica tested. The first pattern of expression is demonstrated by the robusta FRT32 and the arabica T2308. These varieties show relatively high levels of expression at each stage of development. This result was somewhat unexpected, i.e., one robusta being very similar to an arabica expression pattern, but different from other robustas. The second pattern is shown by the two other robusta (FRT05 and FRT64), and in this case, there was a relatively high level of transcription in the early small green stage and then the transcript levels fell significantly at each later stage. The expression pattern for UGE5 is slightly more complicated. Nonetheless, there again appears to be two different groups of expression, one in which there are relatively low levels of UGE5 transcripts at all stages (FRT32 and arabica T2308) and the other two robusta, which show much higher levels. One observation from the quantative transcript expression data for the coffee UGE1 and UGE5 genes is that the levels of UGE1 transcripts are much more significant than the UGE5 transcript levels in the robusta FRT32 and the arabica T2308 and that the reverse is true for the other two robustas. This observation could suggest that these two genes can substitute for one another in the grain.

The expression data for the UGPP, GMPP and PMM genes is presented in FIG. 8 and set forth in Tables 11 (UGPP), 12 (GMPP) and 13 (PMM) below.

TABLE 11

| | FRT32 | | FRT05 | | FRT64 | | T2308 | |
|---|---|---|---|---|---|---|---|---|
| | medium | standard | medium | standard | medium | standard | medium | standard |
| Sample | RQ | deviation | RQ | deviation | RQ | deviation | RQ | deviation |
| G-SG | 0.092 | 0.018 | 0.185 | 0.040 | 0.153 | 0.021 | 0.114 | 0.017 |
| G-LG | 0.110 | 0.019 | 0.073 | 0.011 | 0.134 | 0.005 | 0.324 | 0.036 |
| G-Y | 0.156 | 0.023 | 0.096 | 0.009 | 0.155 | 0.017 | 0.124 | 0.012 |
| G-R | 0.209 | 0.021 | 0.042 | 0.006 | 0.115 | 0.010 | 0.192 | 0.020 |

TABLE 12

| | FRT32 | | FRT05 | | FRT64 | | T2308 | |
|---|---|---|---|---|---|---|---|---|
| | medium | standard | medium | standard | medium | standard | medium | standard |
| Sample | RQ | deviation | RQ | deviation | RQ | deviation | RQ | deviation |
| G-SG | 0.020 | 0.003 | 0.099 | 0.020 | 0.144 | 0.021 | 0.072 | 0.010 |
| G-LG | 0.046 | 0.003 | 0.055 | 0.003 | 0.098 | 0.005 | 0.057 | 0.003 |
| G-Y | 0.084 | 0.007 | 0.030 | 0.002 | 0.075 | 0.010 | 0.009 | 0.002 |
| G-R | 0.016 | 0.002 | 0.017 | 0.003 | 0.040 | 0.008 | 0.012 | 0.001 |

TABLE 13

| | FRT32 | | FRT05 | | FRT64 | | T2308 | |
|---|---|---|---|---|---|---|---|---|
| | medium | standard | medium | standard | medium | standard | medium | standard |
| Sample | RQ | deviation | RQ | deviation | RQ | deviation | RQ | deviation |
| G-SG | 0.004 | 0.001 | 0.036 | 0.010 | 0.033 | 0.003 | 0.018 | 0.002 |
| G-LG | 0.016 | 0.001 | 0.036 | 0.003 | 0.037 | 0.003 | 0.023 | 0.003 |
| G-Y | 0.014 | 0.001 | 0.049 | 0.005 | 0.063 | 0.003 | 0.015 | 0.004 |
| G-R | 0.012 | 0.001 | 0.012 | 0.003 | 0.028 | 0.003 | 0.019 | 0.002 |

Overall, the grain expression data for the four genes indicates that the two more “upstream” genes, UGPP and PMM, are expressed in a relatively uniform manner over the stages of grain development examined. This profile possibly indicates the more housekeeping type function of these two genes. In contrast, the genes downstream (GMPP and UGE) appear to show more development related profiles, suggesting their expression could more closely reflect the actual needs of the galactomannan synthesis and other UDP-galactose and GDP-mannose reactions. For example, the transcript accumulation of GMPP transcripts in the grain is higher at the beginning of the maturation (at the small green stage) and then progressively decreases during the maturation. This could reflect the high demand for GDP-mannose in the galactomannan synthesis, which corresponds well to the increased expression of the ManS 1 gene at the large green and yellow stages (Pre et al., 2008).

C. Comparative Expression Analysis of PMM, GMPP, UGPP and UGE in in Different Tissues and/or Stages of Development of Robusta FRT32 and/or Arabica T2308

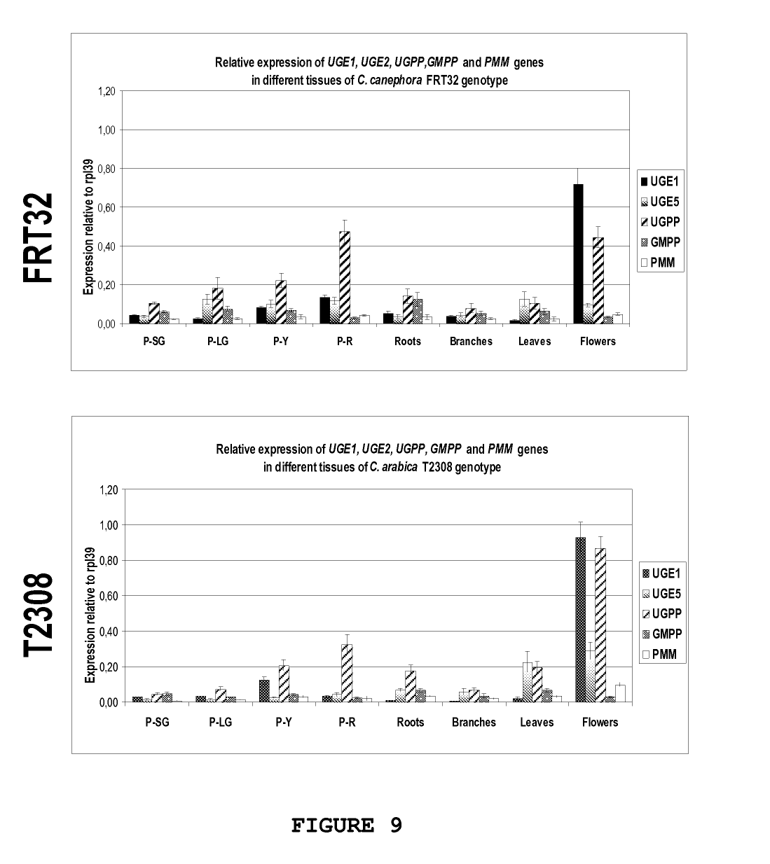

FIG. 9 shows the more global expression data obtained for PMM, GMPP, UGPP and UGE. Clearly, these genes are widely expressed in the plant, reflecting the fact that they are involved in central metabolism, and that except for UGE, are represented by single genes, at least in the Arabidopsis genome. The RQ medium values are shown in Tables 14 and 15.

TABLE 14

| | UGE1 | | UGE5 | | UGPP | | GMPP | | PMM | |
|---|---|---|---|---|---|---|---|---|---|---|
| | RQ | Standard | RQ | Standard | RQ | Standard | RQ | Standard | RQ | Standard |
| Sample | medium | deviation | medium | deviation | medium | deviation | medium | deviation | medium | deviation |
| G-SG | 0.144 | 0.019 | 0.010 | 0.004 | 0.092 | 0.018 | 0.020 | 0.003 | 0.004 | 0.001 |
| G-LG | 0.110 | 0.010 | 0.011 | 0.001 | 0.110 | 0.019 | 0.046 | 0.003 | 0.016 | 0.001 |
| G-Y | 0.176 | 0.017 | 0.013 | 0.001 | 0.156 | 0.023 | 0.084 | 0.007 | 0.014 | 0.001 |
| G-R | 0.279 | 0.017 | 0.035 | 0.004 | 0.209 | 0.021 | 0.016 | 0.002 | 0.012 | 0.001 |
| P-SG | 0.045 | 0.004 | 0.038 | 0.008 | 0.105 | 0.007 | 0.061 | 0.007 | 0.025 | 0.002 |
| P-LG | 0.027 | 0.004 | 0.126 | 0.027 | 0.184 | 0.057 | 0.075 | 0.016 | 0.027 | 0.004 |
| P-Y | 0.085 | 0.007 | 0.102 | 0.019 | 0.223 | 0.036 | 0.068 | 0.008 | 0.035 | 0.011 |
| P-R | 0.133 | 0.013 | 0.117 | 0.016 | 0.475 | 0.062 | 0.031 | 0.006 | 0.045 | 0.004 |
| Roots | 0.054 | 0.012 | 0.036 | 0.010 | 0.143 | 0.037 | 0.126 | 0.034 | 0.035 | 0.013 |
| Branches | 0.038 | 0.007 | 0.045 | 0.010 | 0.079 | 0.024 | 0.054 | 0.009 | 0.026 | 0.004 |
| Leaves | 0.016 | 0.006 | 0.128 | 0.037 | 0.105 | 0.031 | 0.063 | 0.015 | 0.025 | 0.012 |
| Flowers | 0.720 | 0.079 | 0.095 | 0.010 | 0.445 | 0.055 | 0.034 | 0.004 | 0.049 | 0.005 |

TABLE 15

| | UGE1 | | UGE5 | | UGPP | | GMPP | | PMM | |
|---|---|---|---|---|---|---|---|---|---|---|
| | RQ | Standard | RQ | Standard | RQ | Standard | RQ | Standard | RQ | Standard |
| Sample | medium | deviation | medium | deviation | medium | deviation | medium | deviation | medium | deviation |
| G-SG | 0.170 | 0.017 | 0.018 | 0.003 | 0.114 | 0.017 | 0.072 | 0.010 | 0.018 | 0.002 |
| G-LG | 0.588 | 0.032 | 0.062 | 0.004 | 0.324 | 0.036 | 0.057 | 0.003 | 0.023 | 0.003 |
| G-Y | 0.418 | 0.024 | 0.021 | 0.003 | 0.124 | 0.012 | 0.009 | 0.002 | 0.015 | 0.004 |
| G-R | 0.521 | 0.037 | 0.057 | 0.003 | 0.192 | 0.020 | 0.012 | 0.001 | 0.019 | 0.002 |
| P-SG | 0.029 | 0.002 | 0.013 | 0.006 | 0.044 | 0.008 | 0.047 | 0.010 | 0.007 | 0.003 |
| P-LG | 0.033 | 0.002 | 0.013 | 0.005 | 0.074 | 0.011 | 0.027 | 0.002 | 0.012 | 0.002 |
| P-Y | 0.122 | 0.020 | 0.023 | 0.003 | 0.206 | 0.031 | 0.043 | 0.006 | 0.030 | 0.007 |
| P-R | 0.033 | 0.007 | 0.042 | 0.012 | 0.326 | 0.057 | 0.024 | 0.005 | 0.018 | 0.013 |
| Roots | 0.008 | 0.001 | 0.068 | 0.009 | 0.178 | 0.033 | 0.067 | 0.008 | 0.033 | 0.004 |
| Branches | 0.006 | 0.001 | 0.058 | 0.019 | 0.066 | 0.014 | 0.035 | 0.011 | 0.018 | 0.005 |
| Leaves | 0.021 | 0.006 | 0.226 | 0.059 | 0.194 | 0.035 | 0.066 | 0.011 | 0.032 | 0.006 |
| Flowers | 0.930 | 0.085 | 0.289 | 0.050 | 0.868 | 0.064 | 0.029 | 0.003 | 0.096 | 0.012 |

For robusta, all the genes are expressed in the pericarp at relatively similar levels, although UGPP seems to be significantly higher in yellow and especially at the red stages, suggesting an increased flow of the related metabolites may occur at these stages of fruit ripening, causing increased UDP-Glu levels, perhaps causing increased sucrose synthesis in fruit, or increased Glu-1-P production. Expression in arabica followed the same general pattern, except that the levels of UGE5 were slightly lower than seen for the robusta. Similar expression patterns were also seen for the roots, branch, leaf, and flower. A noteworthy aspect of these expression data is the very high levels of expression seen for the UGE1 and UGPP genes in the flowers, suggesting that there could be a high level of UDP-galactose flux (forward or backward) in the flowers at this stage of development.

In A. thaliana, UGEs were shown to be necessary for the good development of young plantlets (Rösti et al., 2007, supra). Also, the UGPP1 gene from O. sativa has been shown to be expressed throughout the plant, with a peak of expression in florets, especially in pollen during anther development (Chen et al., 2007, supra). UGPP1 silencing by RNA interference or cosuppression resulted in male sterility and in various pleiotropic developmental abnormalities, suggesting that this UGPase plays important roles in plant growth and development. It is likely the orthologues of coffee have similar functions.

The present invention is not limited to the embodiments described and exemplified above, but is capable of variation and modification within the scope of the appended claims.

1. A nucleic acid molecule isolated from Coffea spp. comprising a coding sequence that encodes a galactomannan precursor synthesis enzyme selected from the group consisting of UDP-glucose pyrophosphorylase (UGPP), GDP-mannose pyrophosphorylase (GMPP), phosphomannomutase (PMM), and UDP-glucose 4-epimerase (UGE).

2. The nucleic acid molecule of claim 1, wherein the galactomannan precursor synthesis enzyme comprises an amino acid sequence greater than about 80% identical across its entirety to that of any one of SEQ ID NOs: 6-10, as determined by BLAST comparison.

3. The nucleic acid molecule of claim 1, wherein the galactomannan precursor synthesis enzyme comprises any one of SEQ ID NOs: 6-10.

4. The nucleic acid molecule of claim 1, comprising any one of SEQ ID NOs: 1-5.

5. The nucleic acid molecule of claim 1, wherein the coding sequence comprises a molecule/gene selected from the group consisting of an open reading frame of a gene, or an mRNA molecule produced by transcription of the gene, and a cDNA molecule produced by reverse transcription of the mRNA molecule.

6. A vector comprising the coding sequence of the nucleic acid molecule of claim 1.

7. The vector of claim 6, wherein the coding sequence of the nucleic acid molecule is operably linked to a promoter selected from the group consisting of a constitutive promoter, an inducible promoter, or to a tissue specific promoter.

8. A fertile plant produced from a plant cell transformed with the vector of claim 7.

9. A method of modulating extractability of solids from coffee seeds, comprising modulating production or activity of one or more galactomannan precursor synthesis enzymes within coffee seeds to result in altered galactomannan content of the coffee seeds, wherein the galactomannan precursor synthesis enzyme is selected from the group consisting of UDP-glucose pyrophosphorylase (UGPP), GDP-mannose pyrophosphorylase (GMPP), phosphomannomutase (PMM), and UDP-glucose 4-epimerase (UGE).

10. The method of claim 9, comprising increasing production or activity of at least one galactomannan precursor synthesis enzyme selected from the group consisting of UGPP, GMPP, PMM, and UGE within the coffee seeds.

11. The method of claim 10, comprising increasing expression of a gene encoding at least one galactomannan precursor synthesis enzyme selected from the group consisting of UGPP, GMPP, PMM, and UGE within the coffee seeds.

12. The method of claim 11, comprising introducing one or more transgenes encoding at least one galactomannan precursor synthesis enzyme selected from the group consisting of UGPP, GMPP, PMM, and UGE into the coffee plant for expression within the seeds.

13. The method of claim 9, comprising decreasing production or activity of at least one galactomannan precursor synthesis enzyme selected from the group consisting of UGPP, GMPP, PMM, and UGE within the coffee seeds.

14. The method of claim 13, comprising decreasing expression of a gene encoding at least one galactomannan precursor synthesis enzyme selected from the group consisting of UGPP, GMPP, PMM, and UGE within the coffee seeds.

15. The method of claim 14, comprising introducing into the coffee plant for expression within the seeds one or more polynucleotides encoding an inhibitor of translation of at least one galactomannan precursor synthesis enzyme selected from the group consisting of UGPP, GMPP, PMM, and UGE.

16. The vector of claim 7, wherein the coding sequence of the nucleic acid module is a seed specific promoter.

17. A fertile plant produced from a plant cell transformed with the vector of claim 7 and the plant is a coffee plant.


SEQUENCE LISTING (53 sequences)

SEQ ID NO: 1 - DNA, 1750 bp, Coffea canephora
catcatcctcctcctgatcccttctagaacacttccattccctattattctctgtcgttttctagatcggagatttgcaaaataaattgttgaaaatcatcaatggcaactgccgcgactctaaatccagctgaggctgagaagctcgagaagcttaaagctgctacagcggcgctcaatcaaatcagtgaaaacgagaaatctggattcatcaacctcatcgctcgctatcttagcggcgaagcgcagtatgttgactggagcaagatccagacacccacagatgaggttgtggtgccttatgacaccttagcacccgtgtctgaagatcctgcagaaacaaagaagcttttggacaaacttgttgtgttaaagctcaatggtggcttgggcacgacaatgggttgcactggtccaaagtcggtcattgaggttcgcaatggtttgacattccttgacttgatagttatccaaatcgagacactcaacaagaagtacggatgcaacgtacccttgcttctaatgaactccttcaacacacatgatgacacgctgaagattgtagaaaagtatgccaaatcaaacatcgaaattcatacattcaaccagagtcaataccctcgattggtggttgaagatttcatgccacttccatctaaaggaaatactggaaaggatgcatggtatcctcctggccatggtgatgtgttcccatccttgatgaatagtggcaaacttgatgctttattatcacaggggaaggaatatgtatttgttgcaaattcagataacttgggtgcagttgttgatttaaaaatcttaaaccacttgatcagtaacaagaacgaatactgcatggaggtcactccaaaaacattggctgatgttaaaggtggtacgctcatttcttatgaaggaagagtgcagctcctggaaatagctcaagttcctgatgaacatgtgaacgaattcaagtcaatagagaagttcaaaattttcaataccaacaacttatgggtgaacttgaaagcaatcaaaagacttgtacaagaaagtgcactgaagatggagattattccaaatccaaaggaagtagatggggtgaaagttcttcaacttgaaactgctgctggcgctgcaataaggttctttgatcgtgctattggcatcaatgttcctcgatctcgattcctcccagtaaaggcaacttctgatctgcttctggtccagtccgacctgtacactttgtctgatgatggctatgtggtccgcaacgaggctaggaaaaatccaactaacccaactattgaattgggacctgaatttaagaaggtcggcaacttcttaagtcgtttcaagtctattcccagtattgttgaactagatagcctgaaggtgagcggcgatgtgtggtttggatctggcatcaccctgaaggggaaagtgacaataactgcaaaacctggaaccaagttagaaattcccgacagagctgtaattgctgacaaggttataaatggccctgaggatatttaagaggtctaattctctggaagtgatgcatagttttgcagaagcgatggggactttttggttgtgatgacacttgtttaactttctatgggttatgtagtcacatatggggcttgctttcttggttgcctgaatagcacatatatagttgacggtaaaaaataaatggacggttttgttccgcaaaaaaaaaaaaaaaaaaaaaaaaaaaaaaaaa

SEQ ID NO: 2 - DNA, 1576 bp, Coffea canephora
atttgattttattattgctcaacccagaagcggaagaaacttccttccgagtcagctcgctttctgactcgcttttctctccatttccaattcgtatagtctcagttctctgttcacataacacgataaccaggagcatatcatttcaggatgaaggcacttattcttgttggaggctttgggactcgtttgaggccactgacactaagtgtccctaagccactagtggattttgctaataaaccaatgatcttgcatcagattgaggctcttaaggctattggagtgactgaagtggttcttgcaatcaactaccagccagaagtgatgctgaatttcctgaaagacttcgaagcaaagcttgggatcaggatcacatgctcacaagagactgagccacttggaactgcaggcccccttgctttggctagggacaagctggcagatggttctggcgagccattttttgttctgaacagtgatgttatcagtgaatatccactcaaagagatgattgaattccacaaatcgcatgggggtgaggcttccattatggtaactaaggtggatgaaccttcaaaatatggggttgttgtcttggaagaagccactggacaggtggagaggtttgtggagaagcccaaactatttgttggcaacaaaattaatgctggcatttaccttctgaacccttctgttcttggtcgaattaaattaaggcccacttcaattgagaaagaggttttcccaaaaattgcagcagagaaaatgctttatgctatggttttgcctgggttctggatggacattggacagccgagggattacatcactggcttgcggttgtatctggattccttgaggaagaaagatgcctcaaaattggcttctggaactcatattgttggaaatgttcttgtgcatgagagtgccaaaataggagagggttgcttgattggacccgatgttgcaattggccctggatgtgtggttgaggcaggtgttagactctcacgctgcacggtaatgcgtggagttcgcatcaagaaacatgcctgcatctcaagcagtatcattggctggcactcgacagttgggcagtgggctcgtgtggagaacatgaccattttgggagaagatgttcatgtttgtgatgaaatctacagcaacgggggtgtagttttgccccacaaggagattaaatcaagcatcttgaagccagagattgttatgtgaaactttgttgctagctctaggtgatagagacttcatatttctcatatttatgtgtgttctttcccccccttttttttggctcctcttcaaatgtaaaatttagcctgagcaagtttgagtgacttgagcaatttggtgcaaggttgatgtctgttgtgaaaggatacaagtataggtcactggtgtgaaatatgtaatattttctgtatatagaaataaattattggttgttgaagaacgaaatgtaattttagcttcccgttaaactttcaataagatgctagcattgtgtgtaaaaaaaaaaaaaaaaaaaaaaaaaaaaaaaaaaaaaaaaaaaaaa

SEQ ID NO: 3 - DNA, 1218 bp, Coffea canephora
gctgctttgaagacgacttcatccggcttcccaccgcaactgacagagaaaagtgaaacggttgtgggcctgctagagatcccacgttcttcagctctcaagtctcaaccttcaactttatatataccgaactctccttgctgaagaatccaacgtccccactcggagccctcattcgtcaccaatctccaccttttttacagagaatggctgggatcaaacctggtgtgctagctctgtttgatgtggatggcacccttaccgccccgcgaaaggcagctactcctgaaatgttacaattcatgcagaaattgaggaaggttgtggctgttggtgttgttgggggttccgatctcgttaagatatcagaacagcttggaaagacagttataactgattacgattatgttttttccgagaatggcctggtggcttataagggaggaaagctgattggaacacagagtttgaagtcatttcttggtgaagaaaagctcaaggagtttattaacttcacgctccattatattgctgacttggacatcccaataaaaaggggtacattcattgagtttcgaagtggtatgcttaatgtgtccccaattgggaggaattgtagtcaggaagaaagagatgagtttgaaaaatatgacaaggttcacaatatacgtccaaagatggtctcagtgctaagggagaaatttgcacatcttaaccttaccttttcaattggaggacaaattagttttgatgttttccctcaaggatgggataagacatattgtttgagatacgttgatgagtttccggagattcacttctttggtgacaagacatacaagggcggaaatgattccgagatctatgaatctgagagaaccgtgggacacacagttactagcccggatgatacagtaaagctgtgttcaagtcttttcctgggctaggaggttgcagattcttgaccatgttgcatacacttggcttttcaattgtttctaatcatttataaagtggtcttcagttgtgaaactttccatctaccctgtattaccattcttatgtatgttcaagttgtaaaattttgccttgtaatctgtggatttagtaaaaattcctttcaaacaataaccttttgctaaacagtgtatttattctgtgctatgtaatgaagaataagaggaagaaagacaaggcctttttctaaaaaaaaaaaaa

SEQ ID NO: 4 - DNA, 1424 bp, Coffea canephora
  misc_feature (1371)..(1372): n is a, c, g, or t
gtgccttggctcatctttttatccgttgcttcgaacagatatctcagtttcttcatactatttctttccatttccatcgatttcccagtattatactcttgaacccttttcaagcctccccatttcagacacttttttgtttttcacaggacaagaagggttcttttttgctttacttgtgaaagaatggccgtgggagtggaagataggattttggtgacaggaggggctggtttcattggaactcatactgttctgcagctattgaaagaaggttttaaggtttcaatcattgacaacctcgataactccgtcgaagaggctgttcttcgagtaagagagttagcgggaccccagttgtctaaaaatcttgatttctatctgggtgatattagaaataaggaggatttggaggagatattttcaaaaaacaagtttaatgctgttatacactttgctggcctgaaagctgttggggagagtgttgcttatcctatgcgctattttgacaacaatttggctggatcaatcaatttgtacatgaccatggcaaagtacaattgcaagaagttggttttctcttcatctgccactgtatatggccaacctgaaaaaattccatgtgttgaagattttgatttgaaggctatgaatccctatggccgcacaaagctattcctggaagaaattgctggagatattcaaaaggcagatccagaatggaaaattatcttgctaagatatttcaaccctgttggggctcatgagagtggtaggcttggagaagatcctaagggtatcccaaacaatctcatgccttacatagaacgagtagctgttggtagactacctgagttaaatgtttatggtcatgattatcccacacctgatggtagtgcaatacgagactatatccatgtgatggatttggcagatggtcatgttgctgccttgcagaagctttttcgaatggattttaaaggttgtcatgcttacaacttggggactggccaaggtacatctgtcctcgaaatggttgctgctttcaaaagagcatctggaaaggacatccccatcaaactgtgtccaagaaggccaggagatgccactgcagtttatgcatcaacagagaaagctgagaaagagcttggttggaaggcaaagtacaacatagatgatatgtgcagcgatcagtggaagtgggcaagcctgaatccatggggttacgagggcaagccttgactcttgatacctctttatcccaactttatcagtagttgttgttcaattcctagcattgtgttaatactatgaatatttggaagcaaaatcaactcaaacgtgatggtccttctgttcattgaacgaagnncaaatcactcgaaaatncactctgatttcagatctccaaaaaaaaaaaaaaa

SEQ ID NO: 5 - DNA, 1434 bp, Coffea canephora
gttcctataaatattctgaagtctcttttttttctgctagactggcagagaaaggagtgcagatagttttgtatactggttaggctcgggcaaaagattgagcggaaaaacgatgccggagaagatgaatatattggtgacgggaggggcagggtatataggaagccatacagttcttcaattgctgttgggagggtacaagactgttgtggttgataatttggacaattcttccgatgttgccctcaagagggtccgagaactcgccggtgaacatggctccaacctcacctttcataagatggatcttcgtgacaaggttgcacttgaaaatctttttgtttctgaaaagtttgatgcagtaatccattttgctggactgaaagcagtaggtgaaagtgtgcaaaaaccgttgatgtactatgacaacaatcttgtcggtacaattaccctactagaagtcatggctgctcatggatgtaaaaagcttgtgttttcatcatcggctactgtttatggttggccaaagggggtcccttgcactgaggaatttcccttatgtgctgtaaatccatatggacgaaccaagctttttattgaagaaatctgtcgtgatgtgtatggttcagactctgaatggaaaatcatattgctgcggtacttcaatccggttggtgcacatcctagtggttatattggtgaagatccacgtggaattccaaacaatctcatgccttttgtgcaacaagtagctgttggtaggagacctgctctgacagtttttggaactgattactcaacgaaggatggtactggggtgcgtgactacatccatgttgtggacttagcagatggtcatatagcagcagttaataagctgtctgatccttctatagggtgcgaagtgtacaacttaggaactgggaaaggaacttctgtcttggaaatggtagaagcatttgagaaggcatcaagaaagaaaattccactggtgaaagctggtcgccgggctggtgatgcagagattgtatatggatccacagataaggcagaacatgaactgaactggaaggcaaaatatggcatagaggagatgtgccgagaccagtggaactgggctagcaagaatccttatggctacggatcacctgattctaccgattgattggattctgacattacatcctagagtcaccacccatttgaagagctgtttgctacagagtccaaatgcgtagagctgtccctttttttgttttgttttctttctttttgagtcctttgtaggttgggtaggcagatgagataggattgcagtttaacaataataatgataccagggatgtcatatgcatgtaattctaacatggctcactgctgctgaatatatgatgaacttatatcatcttttataaaaaaaaaaaaaaaaaaaaa

SEQ ID NO: 6 - protein, 477 aa, Coffea canephora
MATAATLNPAEAEKLEKLKAATAALNQISENEKSGFINLIARYLSGEAQYVDWSKIQTPTDEVVVPYDTLAPVSEDPAETKKLLDKLVVLKLNGGLGTTMGCTGPKSVIEVRNGLTFLDLIVIQIETLNKKYGCNVPLLLMNSFNTHDDTLKIVEKYAKSNIEIHTFNQSQYPRLVVEDFMPLPSKGNTGKDAWYPPGHGDVFPSLMNSGKLDALLSQGKEYVFVANSDNLGAVVDLKILNHLISNKNEYCMEVTPKTLADVKGGTLISYEGRVQLLEIAQVPDEHVNEFKSIEKFKIFNTNNLWVNLKAIKRLVQESALKMEIIPNPKEVDGVKVLQLETAAGAAIRFFDRAIGINVPRSRFLPVKATSDLLLVQSDLYTLSDDGYVVRNEARKNPTNPTIELGPEFKKVGNFLSRFKSIPSIVELDSLKVSGDVWFGSGITLKGKVTITAKPGTKLEIPDRAVIADKVINGPEDI

SEQ ID NO: 7 - protein, 361 aa, Coffea canephora
MKALILVGGFGTRLRPLTLSVPKPLVDFANKPMILHQIEALKAIGVTEVVLAINYQPEVMLNFLKDFEAKLGIRITCSQETEPLGTAGPLALARDKLADGSGEPFFVLNSDVISEYPLKEMIEFHKSHGGEASIMVTKVDEPSKYGVVVLEEATGQVERFVEKPKLFVGNKINAGIYLLNPSVLGRIKLRPTSIEKEVFPKIAAEKMLYAMVLPGFWMDIGQPRDYITGLRLYLDSLRKKDASKLASGTHIVGNVLVHESAKIGEGCLIGPDVAIGPGCVVEAGVRLSRCTVMRGVRIKKHACISSSIIGWHSTVGQWARVENMTILGEDVHVCDEIYSNGGVVLPHKEIKSSILKPEIVM

SEQ ID NO: 8 - protein, 246 aa, Coffea canephora
MAGIKPGVLALFDVDGTLTAPRKAATPEMLQFMQKLRKVVAVGVVGGSDLVKISEQLGKTVITDYDYVFSENGLVAYKGGKLIGTQSLKSFLGEEKLKEFINFTLHYIADLDIPIKRGTFIEFRSGMLNVSPIGRNCSQEERDEFEKYDKVHNIRPKMVSVLREKFAHLNLTFSIGGQISFDVFPQGWDKTYCLRYVDEFPEIHFFGDKTYKGGNDSEIYESERTVGHTVTSPDDTVKLCSSLFLG

SEQ ID NO: 9 - protein, 351 aa, Coffea canephora
MAVGVEDRILVTGGAGFIGTHTVLQLLKEGFKVSIIDNLDNSVEEAVLRVRELAGPQLSKNLDFYLGDIRNKEDLEEIFSKNKFNAVIHFAGLKAVGESVAYPMRYFDNNLAGSINLYMTMAKYNCKKLVFSSSATVYGQPEKIPCVEDFDLKAMNPYGRTKLFLEEIAGDIQKADPEWKIILLRYFNPVGAHESGRLGEDPKGIPNNLMPYIERVAVGRLPELNVYGHDYPTPDGSAIRDYIHVMDLADGHVAALQKLFRMDFKGCHAYNLGTGQGTSVLEMVAAFKRASGKDIPIKLCPRRPGDATAVYASTEKAEKELGWKAKYNIDDMCSDQWKWASLNPWGYEGKP

SEQ ID NO: 10 - protein, 350 aa, Coffea canephora
MPEKMNILVTGGAGYIGSHTVLQLLLGGYKTVVVDNLDNSSDVALKRVRELAGEHGSNLTFHKMDLRDKVALENLFVSEKFDAVIHFAGLKAVGESVQKPLMYYDNNLVGTITLLEVMAAHGCKKLVFSSSATVYGWPKGVPCTEEFPLCAVNPYGRTKLFIEEICRDVYGSDSEWKIILLRYFNPVGAHPSGYIGEDPRGIPNNLMPFVQQVAVGRRPALTVFGTDYSTKDGTGVRDYIHVVDLADGHIAAVNKLSDPSIGCEVYNLGTGKGTSVLEMVEAFEKASRKKIPLVKAGRRAGDAEIVYGSTDKAEHELNWKAKYGIEEMCRDQWNWASKNPYGYGSPDSTD

SEQ ID NO: 11 - protein, 477 aa, Solanum tuberosum
MATAATLSPADAEKLNNLKSAVAGLNQISDNEKSGFINLVGRYLSGEAQHIDWSKIQTPTDEVVVPYDKLAPLSEDPAETKNLLDKLVVLKLNGGLGTTMGCTGPKSVIEVRNGLTFLDLIVKQIEALNAKFGCSVPLLLMNSFNTHDDTLKIVEKYANSNIDIHTFNQSQYPRLVTEDFAPLPCKGNSGKDGWYPPGHGDVFPSLMNSGKLDALLAKGKEYVFVANSDNLGAIVDLKILNHLILNKNEYCMEVTPKTLADVKGGTLISYEGKVQLLEIAQVPDEHVNEFKSIEKFKIFNTNNLWVNLSAIKRLVEADALKMEIIPNPKEVDGVKVLQLETAAGAAIKFFDRAIGANVPRSRFLPVKATSDLLLVQSDLYTLTDEGYVIRNPARSNPSNPSIELGPEFKKVANFLGRFKSIPSIIDLDSLKVTGDVWFGSGVTLEGKVTIAAKSGVKLEIPDGAVIANKDINGPEDI

SEQ ID NO: 12 - protein, 469 aa, Oryza sativa
MAVAADVKLEGLRAATDKLDQISENEKSGFISLVSRYLSGEAEQIEWSKIQTPTDEVVVPYDTLSAAPEDLNETKKLLDKLVVLKLNGGLGTTMGCTGPKSVIEVRNGFTFLDLIVIQIESLNKKYGCNVPLLLMNSFNTHDDTQKIVEKYSNSNIEIHTFNQSQYPRIVTEDFLPLPSKGKTGKDGWYPPGHGDVFPSLNNSGKLDTLLAQGKEYVFVANSDNLGAIVDIKILNHLIHNQNEYCMEVTPKTLADVKGGTLISYEGRVQLLEIAQVPDEHVNEFKSIEKFKIFNTNNLWVNLKAIKRLVEAEALKMEIIPNPKEVDGVKVLQLETAAGAAIRFFEKAIGINVPRSRFLPVKATSDLLLVQSDLYTLVDGFVIRNPARTNPSNPSIELGPEFKKVANFLARFKSIPSIVELDTLKVSGDVWFGSGVTLKGKVTITAKSGKLEIPDGAVLENKDINGPEDL

SEQ ID NO: 13 - protein, 476 aa, Cucumis melo
MASAATLSPADTEKLSKLKASVSGLTQISENEKSGFINLVSRYLSGEAQHVEWSKIQTPTDEVVVPYDSLAPVPNDPAETKKLLDKLVVLKLNGGLGTTMGCTGPKSVIEVRNGLTFLDLIVIQIENLNSKYGCNVPLLLMNSFNTHDDTQKIIEKYKGSNVDIHTFNQSQYPRLVAEDYLPLPSKGRTDKDGWYPPGHGDVFPSLKNSGKLDALIAQGKEYVFVANSDNLGAVVDLQILNHLIQNKNEYCMEVTPKTLADVKGGTLISYEGKVQLLEIAQVPDEHVNEFKSIQKFKIFNTNNLWVNLKAIKRLVEANALKMEIIPNPKEVDGIKVLQLETAAGAAIRFFDHAIGINVPRSRFLPVKATSDLLLVQSDLYTLVDGFVLRNKARKDPSNPSIELGPEFKKVGNFLSRFKSIPSIIELDSLKVVGDVSFGAGVVLKGKVTISAKPGTKLAVPDNAVIANKEINGPEDF

SEQ ID NO: 14 - protein, 469 aa, Arabidopsis thaliana
MAATTENLPQLKSAVDGLTEMSESEKSGFISLVSRYLSGEAQHIEWSKIQTPTDEIVVPYEKMTPVSQDVAETKNLLDKLVVLKLNGGLGTTMGCTGPKSVIEVRDGLTFLDLIVIQIENLNNKYGCKVPLVLMNSFNTHDDTHKIVEKYTNSNVDIHTFNQSKYPRVVADEFVPWPSKGKTDKEGRYPPGHGDVFPALMNSGKLDTFLSQGKEYVFVANSDNLGAIVDLTILKHLIQNKNEYCMEVTPKTLADVKGGTLISYEGKVQLLEIAQVPDEHVNEFKSIEKFKIFNTNNLWVNLKAIKKLVEADALKMEIIPNPKEVDGVKVLQLETAAGAAIRFFDNAIGVNVPRSRFLPVKASSDLLLVQSDLYTLVDGFVTRNKARTNPSNPSIELGPEFKKVATFLSRFKSIPSIVELDSLKVSGDVWFGSSIVLKGKVTVAAKSGVKLEIPDRAVVENKNINGPEDL

SEQ ID NO: 15 - protein, 361 aa, Solanum tuberosum
MKALILVGGFGTRLRPLTLSVPKPLVEFANKPMILHQIEALKAVGVTEVVLAINYQPEVMLNFLKEFEASLGIKITCSQETEPLGTAGPLALARDKLIDDSGEPFFVLNSDVISEYPFKEMIQFHKSHGGEASLMVTKVDEPSKYGVVVMEESTGQVERFVEKPKLFVGNKINAGFYLLNPSVLDRIQLRPTSIEKEVFPKIAAEKKLYAMVLPGFWMDIGQPRDYITGLRLYLDSLKKHSSPKLASGPHIVGNVIVDESAKIGEGCLIGPDVAIGSGCVIESGVRLSRCTVMRGVRIKKHACISGSIIGWHSTVGQWARVENMTILGEDVHVCDEIYSNGGVVLPHKEIKSSILKPEIVM

SEQ ID NO: 16 - protein, 361 aa, Solanum lycopersicum
MKALILVGGFGTRLRPLTLSVPKPLVEFANKPMILHQIEALKAVGVTEVVLAINYQPEVMLNFLKEFEASLGIKITCSQETEPLGTAGPLALARDKLIDDSGEPFFVLNSDVISEYPFKEMIQFHKSHGGEASLMVTKVDEPSKYGVVVMEESTGQVERFVEKPKLFVGNKINAGFYLLNPSVLDRIQLRPTSIEKEVFPKIAAEKKLYAMVLPGFWMDVGQPRDYITGLRLYLDSLKKHSSPKLASGPHIVGNVIVDESAKIGEGCLIGPDVAIGSGCVIESGVRLSRCTVMRGVRIKKHACISGSIIGWHSTVGQWARVENMTILGEDVHVCDEIYSNGGVVLPHKEIKSSILKPEIVM

SEQ ID NO: 17 - protein, 361 aa, Medicago sativa
MKALILVGGFGTRLRPLTLSVPKPLVDFANKPMILHQIEALKATGVTEVVLAINYQPEVMLNFLKDFEAKLGITISCSQETEPLGTAGPLALARDKLIDDSGEPFFVLNSDVISDYPLKEMIEFHKSHGGEASIMVTKVDEPSKYGVVVMEETTGQVEKFVEKPKLFVGNKINAGIYLLNPSVLDRIELRPTSIEKEIFPKIAAEKKLYAMVLPGFWMDIGQPRDYITGLRLYLDSLRKKSSSKLAGGSNIVGNVIVDETAKIGEGCLIGPDVAIGPGCIVESGVRLSRCTVMRGVRIKKHACISSSIIGWHSTVGQWARVENMTILGEDVHVCDEIYSNGGVVLPHKEIKTNILKPEIVM

SEQ ID NO: 18 - protein, 361 aa, Vitis vinifera
MKALILVGGFGTRLRPLTLSVPKPLVDFANKPMILHQIEALKAVGVSEVVLAINYQPEVMLNFLKEFEAKLGITITCSQETEPLGTAGPLALARDKLIDDSGEPFFVLNSDVISEYPFKEMIEFHKAHGGEASIMVTKVDEPSKYGVVVMEESIGRVDRFVEKPKLFVGNKINAGIYLLNPSVLDRIELRPTSIEKEVFPKIAAEKKLYAMVLPGFWMDIGQPRDYITGLRLYLDSLRKKSSSKLASGAHIVGNVLVDESAKIGEGCLIGPDVAIGPGCVVEAGVRLSRCTVMRGVRIKKHACISSSIIGWHSTVGQWARVENMTILGEDVHVCDEIYSNGGVVLPHKEIKSSILKPEIVM

SEQ ID NO: 19 - protein, 247 aa, Glycine max
MAARRPGLIALFDVDGTLTAPRKVVTPEMLTFMQELRKVVTVGVVGGSDLIKISEQLGSTVTNDYDYVFSENGLVAHKEGKLIGTQSLKSFLGEEKLKEFINFTLHYIADLDIPIKRGTFIEFRSGMLNVSPIGRNCSQEERDEFEKYDKVHNIRPKMVSVLREKFAHLNLTFSIGGQISFDVFPQGWDKTYCLRYLDGFNEIHFFGDKTYKGGNDHEIYESERTVGHTVTSPDDTVKQCKSLFLEN

SEQ ID NO: 20 - protein, 249 aa, Vitis vinifera
MAARKAGLIALFDVDGTLTAPRKVATPQMLEFMRKLRKVITVGVVGGSDLVKISEQLGSSVIDDYDYVFSENGLVAHKDGKLIGTQSLKTFLGEEKLKEIINFTLHYIADLDIPIKRGTFIEFRSGMLNVSPIGRNCSQEERDEFEKYDKIHNIRPKMVSVLREKFAHLNLTFSIGGQISFDVFPQGWDKTYCLRYLDDFPEIHFFGDKTYEAGNDHEIYESERTVGHTVTSPDDTVEQCTALFLAKSS

SEQ ID NO: 21 - protein, 246 aa, Populus trichocarpa
MAVRKPGLIALFDVDGTLTAPRKEATPSMIEFVKELRKVVTIGVVGGSDLSKISEQLGKTVINDYDYVFSENGLVAHKDGKLIGTQSLKSFLGDEKLKEFINFTLHYIADLDIPIKRGTFIEFRSGMLNVSPIGRNCSQEERDEFEKYDKVQNIRPKMVSVLREKFAHLNLTFSIGGQISFDVFPQGWDKTYCLRYLDEFSEIHFFGDKTYKGGNDHEIYESERTVGHTVTSPDDTVEQCKALFFA

SEQ ID NO: 22 - protein, 246 aa, Arabidopsis thaliana
MAAKIPGVIALFDVDGTLTAPRKEATPELLDFIRELRKVVTIGVVGGSDLSKISEQLGKTVTNDYDYCFSENGLVAHKDGKSIGIQSLKLHLGDDKLKELINFTLHYIADLDIPIKRGTFIEFRNGMLNVSPIGRNCSQEERDEFERYDKVQNIRPKMVAELRERFAHLNLTFSIGGQISFDVFPKGWDKTYCLQYLEDFSEIHFFGDKTYEGGNDYEIYESPKTIGHSVTSPDDTVAKCKALFMS

SEQ ID NO: 23 - protein, 351 aa, Arabidopsis thaliana
MGSSVEQNILVTGGAGFIGTHTVVQLLKDGFKVSIIDNFDNSVIEAVDRVRELVGPDLSKKLDFNLGDLRNKGDIEKLFSKQRFDAVIHFAGLKAVGESVENPRRYFDNNLVGTINLYETMAKYNCKMMVFSSSATVYGQPEKIPCMEDFELKAMNPYGRTKLFLEEIARDIQKAEPEWRIILLRYFNPVGAHESGSIGEDPKGIPNNLMPYIQQVAVGRLPELNVYGHDYPTEDGSAVRDYIHVMDLADGHIAALRKLFADPKIGCTAYNLGTGQGTSVLEMVAAFEKASGKKIPIKLCPRRSGDATAVYASTEKAEKELGWKAKYGVDEMCRDQWKWANNNPWGYQNKL

SEQ ID NO: 24 - protein, 351 aa, Arabidopsis thaliana
MGSSVEQNILVTGGAGFIGTHTVVQLLNQGFKVTIIDNLDNSVVEAVHRVRELVGPDLSTKLEFNLGDLRNKGDIEKLFSNQRFDAVIHFAGLKAVGESVGNPRRYFDNNLVGTINLYETMAKYNCKMMVFSSSATVYGQPEIVPCVEDFELQAMNPYGRTKLFLEEIARDIHAAEPEWKIILLRYFNPVGAHESGRIGEDPKGIPNNLMPYIQQVAVGRLPELNVFGHDYPTMDGSAVRDYIHVMDLADGHVAALNKLFSDSKIGCTAYNLGTGQGTSVLEMVSSFEKASGKKIPIKLCPRRAGDATAVYASTQKAEKELGWKAKYGVDEMCRDQWNWANKNPWGFQKKP

SEQ ID NO: 25 - protein, 351 aa, Solanum tuberosum
MGVQCQENILVTGGAGFIGTHTVVQLLNEGFKVTIIDNFHNSVEEAVDRVRELVGPQLSQNLEFHLGDIRNKDDLEKLFSKKEFAAVVHFAGLKAVGESVVQPFLYFENNLIGSITLYSVMAKYNCKKLVFSSSATVYGQPEKVPCVEDFELKAMNPYGRTKLFLEDIARDIQKADQEWNIILLRYFNPVGAHESGKLGEDPKGIPNNLMPYIQQVAVGRLPELNVYGNDYPTPDGTAIRDYIHVMDLADGHVVALQRLLRQNHLGCVAYNLGTGKGKSVLEMVAAFERASGKKIPLKMCPRRPGDATAVYASTEKAEKELGWKAKYGINEMCRDQWKWASQNPWGYQSKP

SEQ ID NO: 26 - protein, 351 aa, Arabidopsis thaliana
MMARNVLVSGGAGYIGSHTVLQLLLGGYSVVVVDNLDNSSAVSLQRVKKLAAEHGERLSFHQVDLRDRSALEKIFSETKFDAVIHFAGLKAVGESVEKPLLYYNNNLVGTITLLEVMAQHGCKNLVFSSSATVYGSPKEVPCTEEFPISALNPYGRTKLFIEEICRDVYGSDPEWKIILLRYFNPVGAHPSGDIGEDPRGIPNNLMPFVQQVAVGRRPHLTVFGNDYNTKDGTGVRDYIHVIDLADGHIAALRKLEDCKIGCEVYNLGTGNGTSVLEMVDAFEKASGKKIPLVIAGRRPGDAEVVYASTERAESELNWKAKYGIEEMCRDLWNWASNNPYGYDSSSEDNSH

SEQ ID NO: 27 - protein, 350 aa, Arabidopsis thaliana
MAKSVLVTGGAGYIGSHTVLQLLEGGYSAVVVDNYDNSSAASLQRVKKLAGENGNRLSFHQVDLRDRPALEKIFSETKFDAVIHFAGLKAVGESVEKPLLYYNNNIVGTVTLLEVMAQYGCKNLVFSSSATVYGWPKEVPCTEESPISATNPYGRTKLFIEEICRDVHRSDSEWKIILLRYFNPVGAHPSGYIGEDPLGVPNNLMPYVQQVAVGRRPHLTVFGTDYKTKDGTGVRDYIHVMDLADGHIAALRKLDDLKISCEVYNLGTGNGTSVLEMVAAFEKASGKKIPLVMAGRRPGDAEVVYASTEKAERELNWKAKNGIEEMCRDLWNWASNNPYGYNSSSNGSSS

SEQ ID NO: 28 - protein, 348 aa, Arabidopsis thaliana
MVGNILVTGGAGYIGSHTVLQLLLGGYNTVVIDNLDNSSLVSIQRVKDLAGDHGQNLTVHQVDLRDKPALEKVFSETKFDAVMHFAGLKAVGESVAKPLLYYNNNLIATITLLEVMAAHGCKKLVFSSSATVYGWPKEVPCTEESPLSGMSPYGRTKLFIEDICRDVQRGDPEWRIIMLRYFNPVGAHPSGRIGEDPCGTPNNLMPYVQQVVVGRLPNLKIYGTDYTTKDGTGVRDYIHVVDLADGHICALQKLDDTEIGCEVYNLGTGKGTTVLEMVDAFEKASGMKIPLVKVGRRPGDAETVYASTEKAERELNWKANFGIEEMCRDQWNWASNNPFGYGSSPNST

SEQ ID NO: 29 - protein, 348 aa, Populus trichocarpa
MAYNILVTGGAGYIGSHTVLQLLLGGYNTVVVDNLDNASDIALKRVKELAGDFGKNLVFHQVDLRDKPALENVFAETKFDAVIHFAGLKAVGESMQKPLLYFNNNLIGTITLLEVMAAHGCKQLVFSSSATVYGWPKEVPCTEEFPLSAANPYGRTKLFIEEICRDIYSSDSEWKITLLRYFNPVGAHPSGYIGEDPRGIPNNLMPYVQQVAVGRRPHLTVFGTDYPTKDGTGVRDYIHVVDLADGHIAALRKLSEANIGCEVYNLGTGKGTSVLEMVAAFEKASGKKIPLVMADRRPGDAETVYAATEKAERDLSWKANYGVDEMCRDQWNWASKNPYGYGSPDGTN

SEQ ID NO: 30 - protein, 362 aa, Solanum tuberosum
  misc_feature (342)..(342): Xaa can be any naturally occurring amino acid
MSKSILVTGGAGYIGSHTVLQLLLGGYKTVVIDSLDNSSEIAVKRVKEIAGEYGSNLSFHKVDLRDKPAVEEIFRSNKFDAVIHFAGLKAVGESVEKPLMYYDNNLIGTITLLEIMAAHGCKRLVFSSSATVYGWPKVVPCTEEFPLSAANPYGRTKLFIEEICRDVQNADSEWKIILLRYFNPVGAHPSGRIGEDPRGIPNNLMPFVQQVAVGRRKELTVYGTDYGTKDGTGVRDYIHVMDLADGHIAALQKLSDPSIGCEVYNLGTGKGTSVLEMVAAFEKASGKKIPMVMSGRRPGDAEIVYAATEKAERELKWKAKYGIEEMCRDQWNWAKKNPYGYXRNSQNLITVTDVYFNCIISS

SEQ ID NO: 31 - protein, 348 aa, Vitis vinifera
MAKTILITGGAGYIGSHTVLQLLLGGFRAVVVDNLDNSSEIAIHRVKELAAEFGDNLVFHKLDLRDKQALEQLFASTNFDAVIHFAGLKAVGESVQKPLLYYDNNLIGTITLLEVMAAHGCKKLVFSSSATVYGWPKEVPCTEEFPLCAANPYGRTKLVIEDICRDIYGSDSEWKIVLLRYFNPVGAHSSGHIGEDPRGIPNNLMPFVQQVAVGRRPALTVFGSDYSTKDGTGVRDYIHVVDLADGHIAALCKLFNSEIGCEVYNLGTGKGTSVLEMVAAFEKASGKKIPLVMAGRRPGDAEIVYASTAKAEKELNWKAKYGISEMCRDQWNWASKNPYGYESSPTQD

SEQ ID NO: 32 - DNA, 21 bp, Artificial sequence
  Synthesized rpl39-F1 primer
gaacaggcccatcccttattg

SEQ ID NO: 33 - DNA, 17 bp, Artificial sequence
  Synthesized rpl39-R1 primer
cggcgcttggcattgta

SEQ ID NO: 34 - DNA, 16 bp, Artificial sequence
  Synthesized rpl39-MGB1 primer
atgcgcactgacaaca

SEQ ID NO: 35 - DNA, 24 bp, Artificial sequence
  Synthesized U348695-F1 primer
gcaaaacctggaaccaagttagaa

SEQ ID NO: 36 - DNA, 25 bp, Artificial sequence
  Synthesized U348695-R1 primer
gccatttataaccttgtcagcaatt

SEQ ID NO: 37 - DNA, 15 bp, Artificial sequence
  Synthesized U348695-MGB1 primer
ttcccgacagagctg

SEQ ID NO: 38 - DNA, 22 bp, Artificial sequence
  Synthesized U352112-F1 primer
gtgtggttgaggcaggtgttag

SEQ ID NO: 39 - DNA, 19 bp, Artificial sequence
  Synthesized U352112-R1 primer
gatgcgaactccacgcatt

SEQ ID NO: 40 - DNA, 16 bp, Artificial sequence
  Synthesized U352112-MGB1 primer
ctctcacgctgcacgg

SEQ ID NO: 41 - DNA, 25 bp, Artificial sequence
  Synthesized U351352-F1 primer
ggtgaagaaaagctcaaggagttta

SEQ ID NO: 42 - DNA, 20 bp, Artificial sequence
  Synthesized U351352-R1 primer
tgggatgtccaagtcagcaa

SEQ ID NO: 43 - DNA, 18 bp, Artificial sequence
  Synthesized U351352-MGB1 primer
aacttcacgctccattat

SEQ ID NO: 44 - DNA, 29 bp, Artificial sequence
  Synthesized U347952-F1 primer
tgttcaattcctagcattgtgttaatact

SEQ ID NO: 45 - DNA, 23 bp, Artificial sequence
  Synthesized U347952-R1 primer
cagaaggaccatcacgtttgagt

SEQ ID NO: 46 - DNA, 14 bp, Artificial sequence
  Synthesized U347952-MGB1 primer
ttggaagcaaaatc

SEQ ID NO: 47 - DNA, 25 bp, Artificial sequence
  Synthesized U352564-F1 primer
tgtatggttcagactctgaatggaa

SEQ ID NO: 48 - DNA, 18 bp, Artificial sequence
  Synthesized U352564-R1 primer
tgtgcaccaaccggattg

SEQ ID NO: 49 - DNA, 19 bp, Artificial sequence
  Synthesized U352564-MGB1 primer
atcatattgctgcggtact

SEQ ID NO: 50 - DNA, 22 bp, Artificial sequence
  Synthesized CcUGPP- forward primer
caccatggcaactgccgcgact

SEQ ID NO: 51 - DNA, 21 bp, Artificial sequence
  Synthesized CcUGPP- reverse primer
ttaaatatcctcagggccatt

SEQ ID NO: 52 - DNA, 22 bp, Artificial sequence
  Synthesized CcUGE2- forward primer
caccatgccggagaagatgaat

SEQ ID NO: 53 - DNA, 22 bp, Artificial sequence
  Synthesized CcUGE2- reverse primer
tcaatcggtagaatcaggtgat